(19) United States
(12) Patent Application Publication
(10) Pub. No.: US 2024/0083846 A1
(43) Pub. Date: Mar. 14, 2024

(54) SMAC MIMETICS FOR TREATMENT OF CANCER, PROCESS FOR PREPARATION AND PHARMACEUTICAL COMPOSITION THEREOF

(71) Applicant: COUNCIL OF SCIENTIFIC & INDUSTRIAL RESEARCH, New Delhi (IN)

(72) Inventors: Wahajul HAQ, Uttar Pradesh (IN); Rafat ALI, Uttar Pradesh (IN); Akhilesh SINGH, Uttar Pradesh (IN); Mushtaq Ahmad NENGROO, Uttar Pradesh (IN); Roshan KATEKAR, Uttar Pradesh (IN); Gajendra SINGH, Uttar Pradesh (IN); Jayanti VAISHNAV, Uttar Pradesh (IN); Mohammad AFSAR, Uttar Pradesh (IN); Manohar SINGH, Uttar Pradesh (IN); Srikanta Kumar RATH, Uttar Pradesh (IN); Dipankar KOLEY, Uttar Pradesh (IN); Durga Prasad MISHRA, Uttar Pradesh (IN); Ravishankar RAMACHANDRAN, Uttar Pradesh (IN); Ravi Sankar AMPAPATHI, Uttar Pradesh (IN); Jiaur Rahaman GAYEN, Uttar Pradesh (IN); Dipak DATTA, Uttar Pradesh (IN)

(21) Appl. No.: 18/267,643

(22) PCT Filed: Dec. 17, 2021
(86) PCT No.: PCT/IN2021/051182

§ 371 Date: Jun. 15, 2023

(30) Foreign Application Priority Data
Dec. 17, 2020 (IN) 202011055682

(51) Int. Cl. C07D 207/34; A61K 45/06; A61P 35/00; C07D 405/04

(52) U.S. Cl. CPC C07D 207/34; A61K 45/06; A61P 35/00; C07D 405/04

(57) ABSTRACT

The present invention relates to novel SMAC mimetic peptidomimetics useful for the treatment of proliferative diseases including cancer in mammals The novel SMAC mimetics are prepared by incorporating (2S,5R)-5-(5-methylfuran-2-yl)pyrrolidine-2-carboxylic acid, a novel unnatural amino acid that imparts exclusively trans amide bond geometry favourable for target protein binding. Here, the novel SMAC mimetic molecule(s) not only show its efficacy in varied cancer types but also demonstrate in vitro and in vivo efficacy against therapy resistant cancer as a single agent. The novel SMAC mimetics disclosed in the present invention binds to BIR-2 and BIR-3 domains of the XIAP and exhibit high anti-proliferative activity against variety of mammalian cancer cell lines that include but not limited to chemotherapy and TRAIL resistant cell lines.

### FIELD OF INVENTION

The present invention is related to SMAC (Second Mitochondria-derived Activator of Caspase) mimetic compounds useful for treatment of proliferative disorder including cancer.

### BACKGROUND OF THE INVENTION

Evasion of apoptosis or ‘programmed cell-death’ is one of the hallmarks of human cancer. Therefore, restoration or induction of apoptosis in cancer cells is an attractive therapeutic strategy. There are multiple endogenous cellular counter acting proteins that regulate intricate balance between cell death and survival. One of the classical examples of such reciprocal regulation within cell is the interaction between SMAC and IAP. The SMAC is a pro-apoptotic protein, sensitizes cells to apoptosis in cancerous cells by antagonizing the activity of IAPs. Therefore, SMAC mimetics have been found as a novel and targeted therapeutic approach to treat cancer (Abraha et. al, World J Gastrointest Oncol. Aug. 15, 2016; 8(8): 583-591). Currently, multiple clinical trials are ongoing for different SMAC mimetics against various cancer types demonstrating its immense importance in cancer therapeutics (Fulda et. al., Clinical Cancer Research. Volume 21, Issue 22, 2015. 5030-5036).

The Inhibitors of Apoptosis Proteins (IAPs) are naturally occurring intra-cellular proteins that suppress caspase-dependent apoptosis. IAPs are the key negative regulators that inhibit the distinct caspases which are critical for initiation and execution of apoptotic pathways. There are eight members in the mammalian IAP family. Among these X-chromosome-linked IAP (XIAP) is perhaps the best characterized member of IAPs family that is known to play a direct role in the regulation of apoptosis. XIAP bind to caspase-3 and caspase-7 via its BIR2 domain and the preceding linker region, respectively. In addition, XIAP also binds to caspase-9 via its BIR3 domain, thereby blocking the dimerization and subsequent activation of caspase-9. Given that fact caspase-3 and -7 play a major role in the implementation of apoptosis in both the extrinsic and intrinsic pathways, and caspase-9 is a critical initiator caspase in the intrinsic pathway, XIAP is the most preferable target to revive the apoptosis. SMAC is the naturally available antagonist of IAP proteins. SMAC is released from the mitochondria into the cytosol upon apoptotic signalling and binds to the BIR3 domain of XIAP via conserved IAP-binding motif (IBM) which contains four amino acid residues (AVPI) that is exposed at the amino-terminus of the mature processed SMAC protein and prevent the interaction of XIAP with caspases (Cong et. al., J. Med. Chem. 2019, 62, 5750-5772).

In addition, Smac also binds to the BIR3 domain of cIAP1 and cIAP2 and as a result enhances their E3 ligase activity which promotes the auto-ubiquitination and proteasomal degradation of cIAP1 and cIAP2. Several small molecule mimetics of AVPI, termed IAP inhibitors are being advanced in clinical trials for the treatment of cancer. The LCL-161 and AT-406 are structurally monovalant whereas Birinapant/TL32711 is a bivalent are among the prominent ones under development.

The major approach used for the designing the SMACs are focussed on the synthesis of conformationally constrained compounds where Xxx-Pro bond is preferably in trans geometry. Yet another consideration was undertaken is the balance of lipophilicity at the C-terminal end or of the linkers for the bivalent molecules. Several efforts are made in the past for the evolution of Smac AVPI tetrapeptide to develop bioavailable Smac mimetics by systematically examination of the role and tolerance to substitution of each amino acids of AVPI (1) peptide.

A library of tetrapeptides using the N-terminal of Smac are prepared with a novel strategy to control trans geometry around the proline residue as a starting point. They replaced the position of each one of the four amino acids with all the natural amino acids. It is found that alanine residue at position 1 of the tetrapeptide is very crucial for activity and binding is greatly diminished if alanine is replaced with any natural amino acids.

Therefore, there is a need in the art for new compounds, which are capable of restoring or inducing apoptosis in cancer cells for treatment of cancer.

### OBJECTIVES OF THE INVENTION

The main objective of the present invention is to develop peptide SMAC mimetics useful as monotherapy as well as in combination with available anti-cancer drugs as safe and effective therapy against various types of cancer.

Another objective of the invention is to develop a process for the synthesis of SMAC mimetics.

Yet another objective of the present invention is to develop formulations of SMAC mimetics suitable for human application.

Still another objective of the present invention is treatment of cancer using SMAC mimetics.

Another objective of the present invention is to provide targeted therapy against treatment resistance cancer by using SMAC mimetics.

Yet another objective of the present invention is to provide SMAC mimetic or IAP antagonist or IAP inhibitor having the capability to potently bind both BIR-2 and BIR-3 domains of XIAP/IAP and having significant bioavailability with robust in-vitro and in-vivo efficacy against therapy resistant refractory cancers.

### SUMMARY OF THE INVENTION

An aspect of the present invention provides a SMAC mimetic compound of Formula-I,

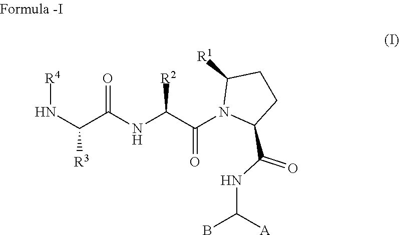

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀ aryl;
B is selected from the group consisting of C₁-C₁₀ aryl, C(O )R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof.

Another aspect of the present invention provides the SMAC mimetic compound selected from the group consisting of

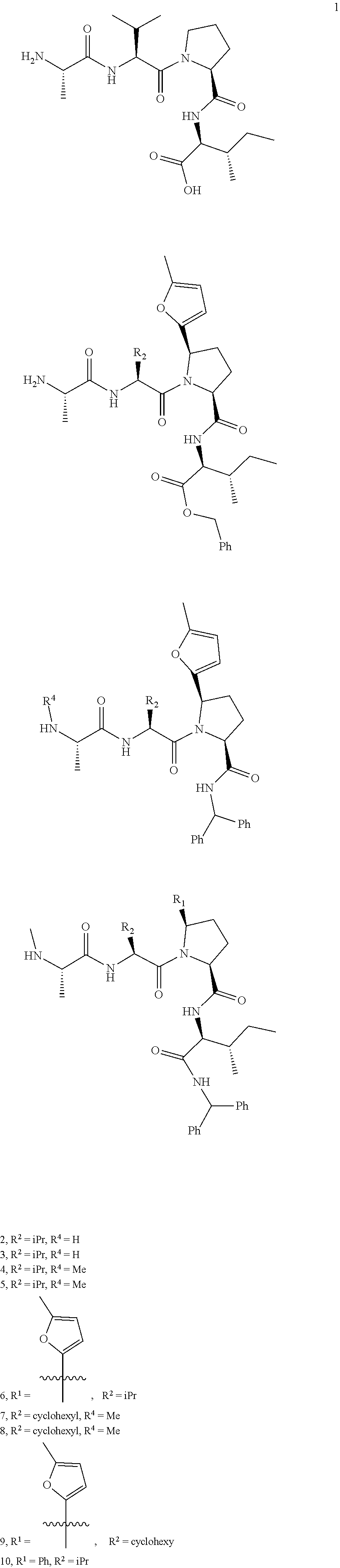

Yet another aspect of the present invention provides a process for preparation of SMAC mimetics compound of Formula-I,

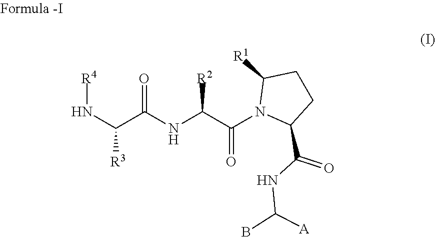

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀aryl;
B is selected from the group consisting of C₁-C₆ aryl, C(O)R⁵ and C(O)N(R⁶)(R⁷);
R⁵ is selected from the group consisting of OH, C₁-C₆ alkoxy or C₆ alkoxyaryl;
R⁶ and R7 are each independently selected from the group consisting of hydrogen, C₆-C₁₀ aryl and C₆-C₁₀ arylalkyl;
comprising the steps of:
a) removing the Boc-group of 2-benzyl 1-(tert-butyl) (2S,5S)-5-(5-methylfuran-2-yl)pyrrolidine-1,2-dicarboxylate by acidolysis using TFA followed by coupling of resulting amine with BocNHCH(R²)COOH in presence of a peptide coupling reagent and a weak base to obtain a compound of formula P1;

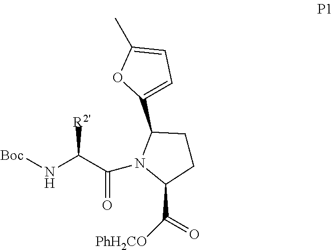

b) removing Boc group from the compound of formula P1 by acidolysis using TFA and coupling the resulting amine with a compound of formula BocN(R^{4′})CH(R^{3′})COOH in the presence of a peptide coupling reagent and a weak base to obtain a compound of formula P2;

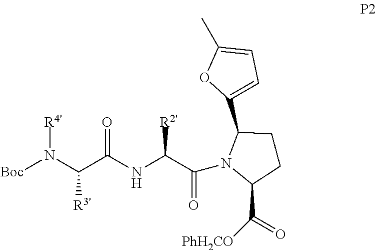

c) catalytic hydrogenation of the compound of formula P2 using Pd-catalyst in presence of a solvent to obtain a free carboxylic acid of formula P3;

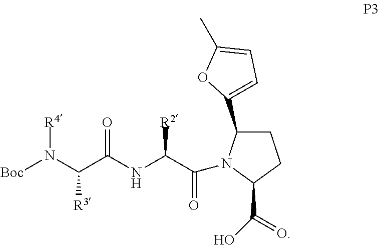

d) coupling the free carboxylic acid of formula P3 with NH₂CH(A)(B) in presence of a peptide coupling reagent and a weak base to obtain the compound of formula I.

Another aspect of the present invention provides a process comprising removal of Boc-group 2-benzyl 1-(tert-butyl) (2S ,5S)-5-(5-methylfuran-2-yl)pyrrolidine-1,2-dicarboxylate followed by the coupling of the resulting amine with Boc-Val-OH to obtain a compound II of which the Boc-group is deprotected followed by its coupling with Boc-Ala-OH to provide a compound III of which the ester is saponified to carboxylic acid followed by its coupling with either H-Ile-OBn or benzhydryl amine followed by its acidolysis to furnish a compound 2 and 3, respectively.

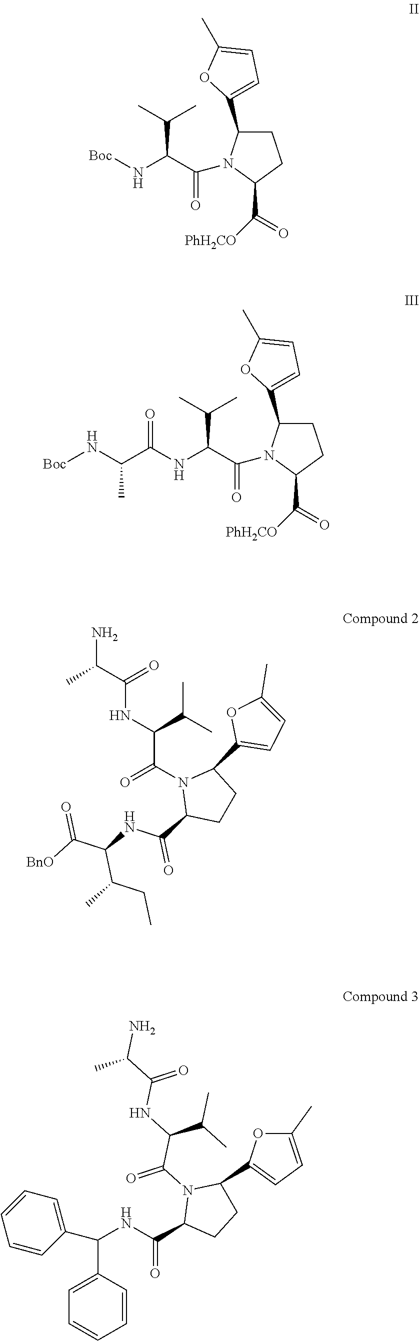

Another aspect of the present invention provides a process comprising removal of Boc-group from a compound II followed by its coupling with Boc-N-Me-Ala-OH to provide 25 a compound V of which the ester is saponified to carboxylic acid followed by its coupling with either H-Ile-OBn or benzhydryl amine or H-Ile-benzhydryl amide followed by its acidolysis to yield compounds 4, 5, and 6, respectively.

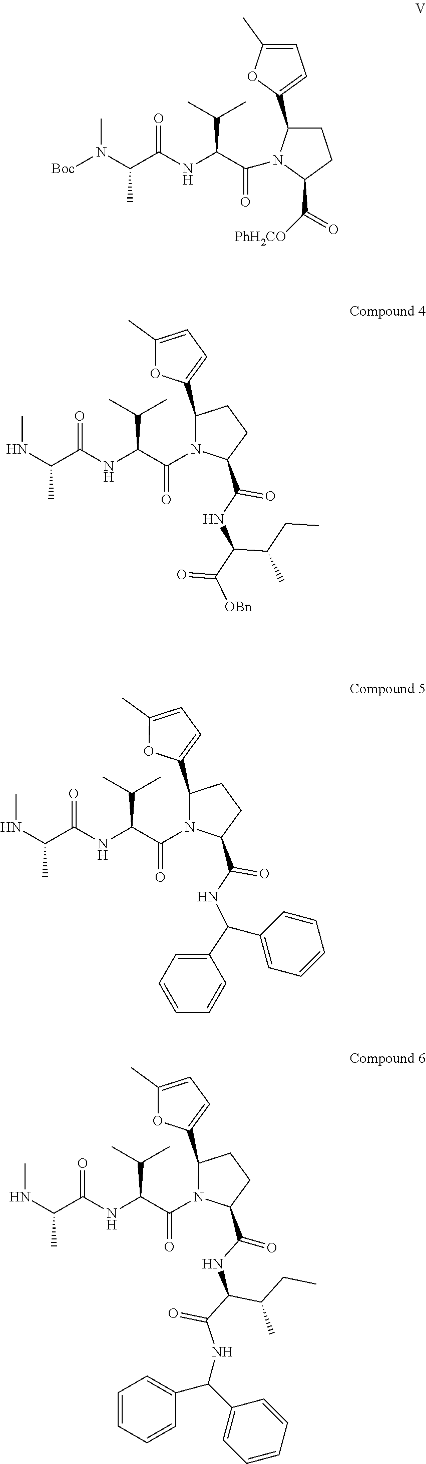

An aspect of the present invention provides a process comprising removal of Boc-group from the intermediate I followed by coupling of the resulting amine with Boc-Chg-OH to obtain a compound VII of which the Boc-group is deprotected followed by coupling with Boc-N-Me-Ala-OH to obtain a compound VIII of which the ester is converted to carboxylic acid followed by its coupling with either H-Ile-OBn or benzhydryl amine followed by its acidolysis to obtain compounds 7, 8 and 9, respectively,

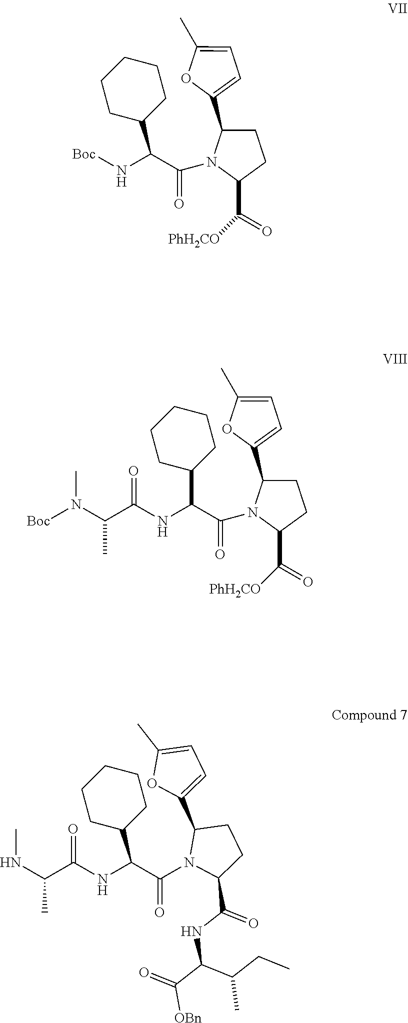

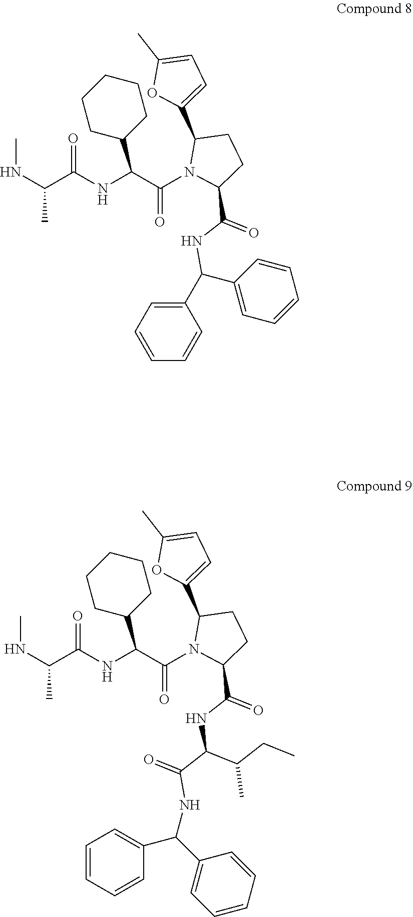 [SMILES: CN[C@@H](C)C(=O)N[C@H](C(=O)N1[C@@H](c2ccc(C)o2)CC[C@H]1C(=O)NC(c1ccccc1)c1ccccc1)C1CCCCC1 ; CC[C@H](C)[C@H](NC(=O)[C@@H]1CC[C@H](c2ccc(C)o2)N1C(=O)[C@@H](NC(=O)[C@H](C)NC)C1CCCCC1)C(=O)NC(c1ccccc1)c1ccccc1]

Another aspect of the present invention provides a process for preparation of SMAC mimetic compound (2S ,5R)-N-((2S ,3S)-1 -(benzhydrylamino)-3-methyl-1 -oxopentan-2-yl)- 1 -((S)-3 -methyl-2-((S)-2-(methylamino)prop anamido)butanoyl)-5-phenylpyrrolidine-2-carboxamide (10), comprising the steps of;
a) saponification and coupling of a compound X with H-Ile-benzhydryl amide in presence of a peptide coupling reagent and a weak base in a solvent to obtain a compound XI;

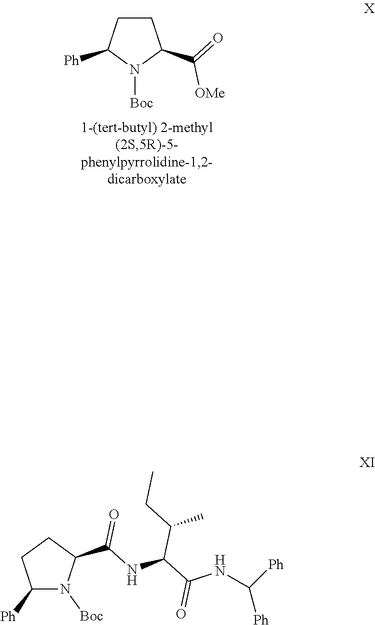 [SMILES: COC(=O)[C@@H]1CC[C@H](c2ccccc2)N1C(=O)OC(C)(C)C ; CC[C@H](C)[C@H](NC(=O)[C@@H]1CC[C@H](c2ccccc2)N1C(=O)OC(C)(C)C)C(=O)NC(c1ccccc1)c1ccccc1]

b) removing the Boc-group from the compound XI by acidolysis using TFA followed by coupling of the resulting amine with Boc-Val-COOH in presence of a peptide coupling reagent and a weak base to obtain a compound of formula XII;

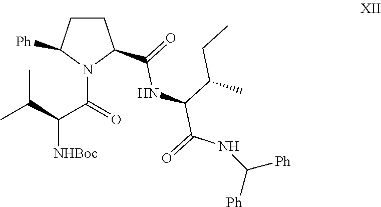 [SMILES: CC[C@H](C)[C@H](NC(=O)[C@@H]1CC[C@H](c2ccccc2)N1C(=O)[C@@H](NC(=O)OC(C)(C)C)C(C)C)C(=O)NC(c1ccccc1)c1ccccc1]

c) removing the Boc-group from the compound XII by acidolysis using TFA followed by coupling of the resulting amine with Boc-N-Me-Ala-OH in presence of a peptide coupling reagent and a weak base to obtain a compound of formula XIII; and

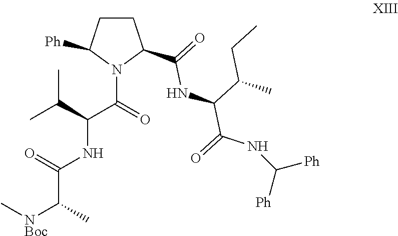 [SMILES: CC[C@H](C)[C@H](NC(=O)[C@@H]1CC[C@H](c2ccccc2)N1C(=O)[C@@H](NC(=O)[C@H](C)N(C)C(=O)OC(C)(C)C)C(C)C)C(=O)NC(c1ccccc1)c1ccccc1]

d) removing the Boc-group from the compound of formula XIII by acidolysis to obtain the compound 10.

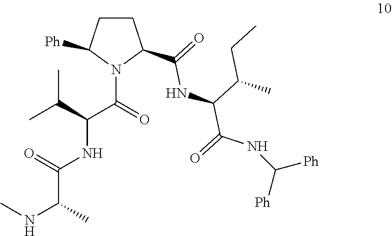 [SMILES: CC[C@H](C)[C@H](NC(=O)[C@@H]1CC[C@H](c2ccccc2)N1C(=O)[C@@H](NC(=O)[C@H](C)NC)C(C)C)C(=O)NC(c1ccccc1)c1ccccc1]

Another aspect of the present invention provides a compound of Formula-I, which inhibits the binding of SMAC protein to Inhibitor of Apoptosis Proteins(IAPs) and is useful in treatment of proliferative diseases including cancer.

Yet another aspect of the present invention provides a pharmaceutical composition comprising SMAC mimetic compound of Formula-I,

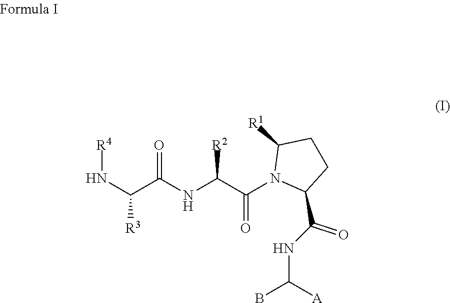

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof and a pharmaceutically acceptable excipient.

Still another aspect of the present invention provides a pharmaceutical composition comprising SMAC mimetic compound of Formula-I,

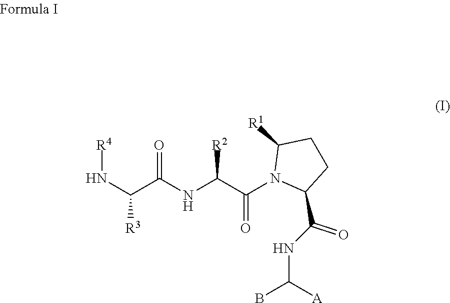

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀ aryl;
B is selected from the group consisting of C₁-C₆ aryl, C(O)R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof, at least one anticancer agent and a pharmaceutically acceptable excipient.

In an aspect of the present invention, the SMAC mimetic compound of Formula I have potent anti-proliferative activity against various mammalian cancer cell lines selected from the group consisting of colon, breast, kidney, prostate, brain, ovary, pancreas, melanoma, liver, leukemia and lymphoma.

In another aspect of the present invention, the SMAC mimetic compound is useful in treatment of therapy resistant, refractory, and metastatic cancers in mammals.

In yet another aspect of the present invention, the SMAC mimetic compound is useful in combination therapies with other anti-proliferative agents selected from the group consisting of TRAIL agonists/MAbs, aromatase inhibitors, epigenetic modulators, kinase inhibitors, alkylating agents, microtubule disrupters, topoisomerase inhibitors, antiangiogenic compounds, Hsp90 inhibitors, mTOR inhibitors, estrogen and androgen antagonists, MMP inhibitors and biological response modifiers.

Another aspect of the present invention provides a method for treating cancer using SMAC mimetic compound of Formula I.

Yet another aspect of the present invention provides a method for treating cancer using SMAC mimetic compounds of Formula-I having the capability to bind to both BIR-2 and BIR-3 domains of XIAP/IAP and having significant bioavailability with robust in-vitro and in-vivo efficacy against therapy resistant refractory cancers.

### BRIEF DESCRIPTION OF THE DRAWINGS:

FIG. 1 shows In Silico Molecular Docking Analysis of C6;

FIG. 2 shows the Mode of Cytotoxic Function of C6;

FIG. 3 demonstrates in vitro target engagement of C6

FIG. 4 shows Synergistic cytotoxic function of C-6 with DR5 ligand TRAIL;

FIG. 5 illustrates the Contribution of target engagement for its cytotoxic function;

FIG. 6 shows the results of Stability studies of C6;

FIG. 7 demonstrates in vivo anti-tumor efficacy of C6 where cisplatin failed to deliver its effect;

FIG. 8 shows C6 treatment that offers robust in vivo efficacy through subcutaneous and oral route of administration;

FIG. 9 shows In-vivo target engagement and tissue distribution of C6.

### ABBREVIATIONS

SMAC: second mitochondrial-derived activator of caspases

TRAIL: tumor necrosis factor-related apoptosis inducing ligand

### DETAILED DESCRIPTION OF THE INVENTION

The invention will now be described in detail in connection with certain preferred and optional embodiments, so that various aspects thereof may be more fully understood and appreciated.

For convenience, before further description of the present disclosure, certain terms employed in the specification, and examples are delineated here. These definitions should be read in the light of the remainder of the disclosure and understood as by a person of skilled in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The present invention relates to SMAC mimetics of Formula-I exhibiting strong anticancer potential in vitro as well as in vivo via apoptotic pathways.

The present invention is directed towards a SMAC mimetic compound of Formula-I,

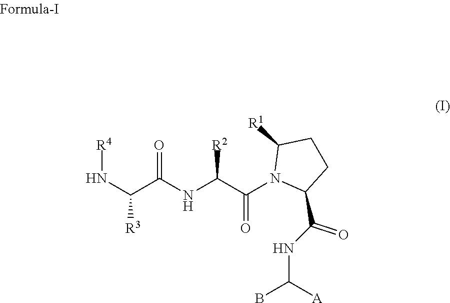

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, Ci-C6 alkoxy and C6 alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof.

In an embodiment of the present invention, there is provided a SMAC mimetic compound of Formula I selected from the group consisting of
L-alanyl-L-valyl-L-prolyl-L-isoleucine (compound 1),
benzyl ((2S,5R)-1-(L-alanyl-L-valyl)-5-(5-methylfuran-2-yl)pyrrolidine-2-carbonyl)-L-isoleucinate (compound 2),
(2S,5R)-1-(L-alanyl-L-valyl)-N-benzhydryl-5-(5-methylfuran-2-yl)pyrrolidine-2-carboxamide (compound 3),
(2S,3S)-benzyl 3-methyl-2-((2S,5R)-1-((S)-3-methyl-2-((S)-2-(methylamino)propanamido)butanoyl)-5-(5-methylfuran-2-yl)pyrrolidine-2-carboxamido)pentanoate (compound 4),
(2S,5R)-N-benzhydryl-1-(methyl-L-alanyl-L-valyl)-5-(5-methylfuran-2-yl)pyrrolidine-2-carboxamide (compound 5),
(2S,5R)-N-((2S,3S)-1-(benzhydrylamino)-3-methyl-l-oxopentan-2-yl)-1-(methyl-L-alanyl-L-valyl)-5-(5-methylfuran-2-yl)pyrrolidine-2-carboxamide (compound 6),
(2S,3S)-benzyl 2-((2S ,5R)-1-((S)-2-cyclohexyl-2-((S)-2-(methylamino)propanamido)acetyl)-5-(5-methylfuran-2-yl)pyrrolidine-2-carboxamido)-3-methylpentanoate (compound 7),
(2S ,5R)-N-benzhydryl-1-((S)-2-cyclohexyl-2-((S)-2-(methylamino)propanamido)acetyl)-5-(5-methylfuran-2-yl)pyrrolidine-2-carboxamide (Compound 8),
(2S,5R)-N-((2S ,3S)-1-(benzhydrylamino)-3-methyl-l-oxopentan-2-yl)-1-((S)-2-cyclohexyl-2-((S)-2-(methylamino)propanamido)acetyl)-5-(5-methylfuran-2-yl)pyrrolidine-2-carboxamide (Compound 9); and
(2S,5R)-N-((2S,3S)-1-(benzhydrylamino)-3-methyl-1-oxopentan-2-yl)-1-(methyl-L-alanyl-L-alanyl)-5-phenylpyrrolidine-2-carboxamide (Compound 10).

Another embodiment of the present invention provides a process for preparation of SMAC mimetics compound of Formula-I,

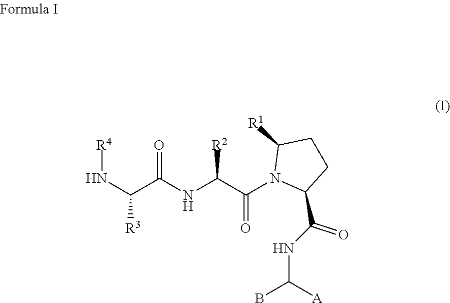

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
comprising the steps of,
i. removing the Boc-group of 2-benzyl 1-(tert-butyl) (2S,5S)-5-(5-methylfuran-2-yl)pyrrolidine-1,2-dicarboxylate by acidolysis using TFA followed by coupling of resulting amine with BocNHCH(R²)COOH in presence of a peptide coupling reagent and a weak base to obtain a compound of formula P1;

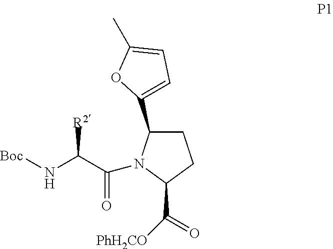

ii. removing Boc group from the compound of formula P1 by acidolysis using TFA and coupling the resulting amine with a compound of formula BocN(R^{4′})CH(R^{3′})COOH in presence of a peptide coupling reagent and a weak base to obtain a compound of formula P2;

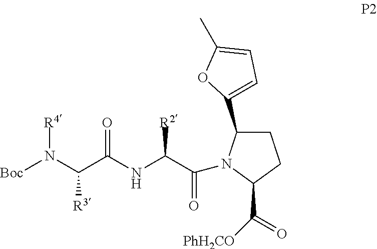

iii. catalytic hydrogenation of the compound of formula P2 using Pd-catalyst in presence of a solvent to obtain a free carboxylic acid of formula P3;

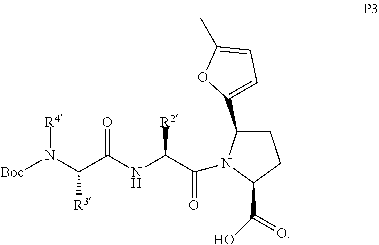

iv. coupling the free carboxylic acid of formula P3 with NH2CH(A)(B) in presence of a peptide coupling reagent and a weak base to obtain the compound of formula I.

In an embodiment of the present invention, there is provided a process for preparation of SMAC mimetics compound of Formula-I, wherein the peptide coupling reagent is selected from the group consisting of HOBt, EDCI and HBTU.

In another embodiment of the present invention, there is provided a process for preparation of SMAC mimetics compound of Formula-I, wherein the weak base is diethylisopropylamine.

In yet another embodiment of the present invention, there is provided a process for preparation of SMAC mimetics compound of Formula-I, wherein the Pd-catalyst is selected from Pd/C, or Pd(OH)₂/C.

In still another embodiment of the present invention, there is provided a process for preparation of SMAC mimetics compound of Formula-I, wherein the solvent is selected from DCM, or DMF for peptide coupling.

In an embodiment of the present invention, there is provided a process for preparation of SMAC mimetics compound of Formula-I, wherein the solvent is selected from MeOH, or EtOAc for catalytic hydrogenation.

In another embodiment of the present invention, there is provided a process of preparation of SMAC mimetic compound 10, comprising the steps of;
(a) saponification and coupling of a compound X with H-Ile-benzhydryl amide in presence of a peptide coupling reagent and a weak base in a solvent to obtain a compound XI;

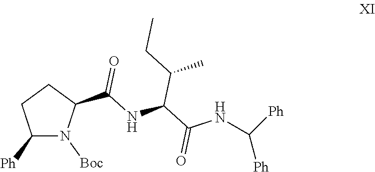 [SMILES: CC[C@H](C)[C@H](NC(=O)[C@@H]1CC[C@H](c2ccccc2)N1C(=O)OC(C)(C)C)C(=O)NC(c1ccccc1)c1ccccc1]

(b) removing the Boc-group from the compound XI by acidolysis using TFA followed by coupling of the resulting amine with Boc-Val-COOH in the presence of a peptide coupling reagent and a weak base to obtain a compound of formula XII;

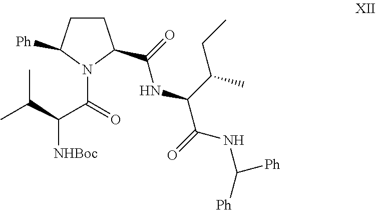 [SMILES: CC[C@H](C)[C@H](NC(=O)[C@@H]1CC[C@H](c2ccccc2)N1C(=O)[C@@H](NC(=O)OC(C)(C)C)C(C)C)C(=O)NC(c1ccccc1)c1ccccc1]

(c) removing the Boc-group from the compound XII by acidolysis using TFA followed by coupling of the resulting amine with Boc-N-Me-Ala-OH in the presence of a peptide coupling reagent and a weak base to obtain a compound of formula XIII; and

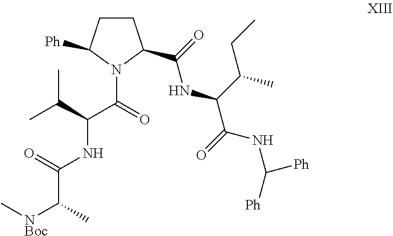 [SMILES: CC[C@H](C)[C@H](NC(=O)[C@@H]1CC[C@H](c2ccccc2)N1C(=O)[C@@H](NC(=O)[C@H](C)N(C)C(=O)OC(C)(C)C)C(C)C)C(=O)NC(c1ccccc1)c1ccccc1]

(d) removing the Boc-group from the compound of formula XIII by acidolysis to obtain the compound 10

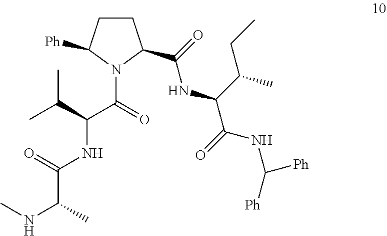 [SMILES: CC[C@H](C)[C@H](NC(=O)[C@@H]1CC[C@H](c2ccccc2)N1C(=O)[C@@H](NC(=O)[C@H](C)NC)C(C)C)C(=O)NC(c1ccccc1)c1ccccc1]

In yet another embodiment of the present invention, there is provided a process of preparation of SMAC mimetic compound 10, wherein the peptide coupling reagent is selected from the group consisting of HOBt, EDCI and HBTU.

In still another embodiment of the present invention, there is provided a process of preparation of SMAC mimetic compound 10, wherein the solvent is selected from DCM, or DMF.

In another embodiment of the present invention, there is provided a process of preparation of SMAC mimetic compound 10 wherein the weak base is diethylisopropylamine.

In yet another embodiment of the present invention, there is provided a process of preparation of SMAC mimetic compound 10, wherein the reagent for acidolysis is TFA.

In another embodiment of the present invention, there is provided a SMAC mimetic compound of Formula I, wherein the compound inhibits binding of Smac protein to Inhibitor of Apoptosis Proteins(IAPs) and is useful in treatment of proliferative diseases including cancer.

Yet another embodiment of the present invention provides a pharmaceutical composition comprising SMAC mimetic compound of Formula-I,

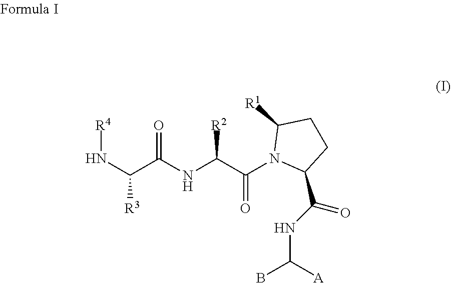

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₆ cycloalkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof and a pharmaceutically acceptable excipient.

Still another embodiment of the present invention provides a pharmaceutical composition comprising SMAC mimetic compound of Formula-I,

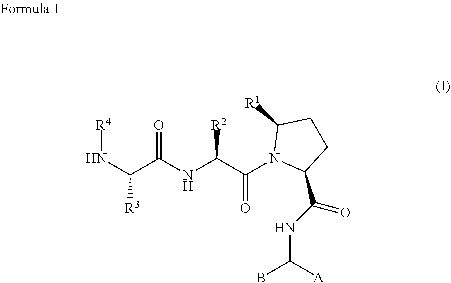

wherein,
R¹ is selected from the group consisting of hydrogen, and unsubstituted or substituted heteroaryl or C₆-C₁₀ aryl;
R², R³ and R⁴ are each independently selected from the group consisting of H, C₁-C₆ alkyl and C₄-C₈ cycloalkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl.

In another embodiment of the present invention, there is provided a SMAC mimetic compound of Formula I having potent anti-proliferative activity against mammalian cancer cell lines selected from the group consisting of colon, breast, kidney, prostate, brain, ovary, pancreas, melanoma, liver, leukemia and lymphoma.

In yet another embodiment of the present invention, there is provided a SMAC mimetic compound of Formula I, wherein the compound is useful in treatment of therapy resistant, refractory, and metastatic cancers in mammals.

In still another embodiment of the present invention, there is provided a SMAC mimetic compound of Formula I, wherein the compound is useful in combination therapies with other anti-proliferative agents selected from the group consisting of TRAIL agonists/MAbs, aromatase inhibitors, epigenetic modulators, kinase inhibitors, alkylating agents, microtubule disrupters, topoisomerase inhibitors, antiangiogenic compounds, Hsp90 inhibitors, mTOR inhibitors, estrogen and androgen antagonists, MMP inhibitors and biological response modifiers.

Still another embodiment of the present invention provides a method for treating cancer using SMAC mimetic compounds.

Accordingly, the present invention relates to SMAC mimetic peptidomimetic compounds of formula-I, useful for the treatment of cancer as a mono and combination therapy where chemotherapy fails to deliver its effect. The SMAC mimetics peptidomimetics compounds 2-9 are prepared by incorporating (2S,5R)-5-(5-methylfuran-2-yl)pyrrolidine-2-carboxylic acid and compound 10 is prepared by incorporating (2S,5R)-5-phenylpyrrolidine-2-carboxylic acid residue, respectively.

The present invention provides a process for preparation of SMAC mimetic compound of Formula-I as described in Scheme A and Scheme B.

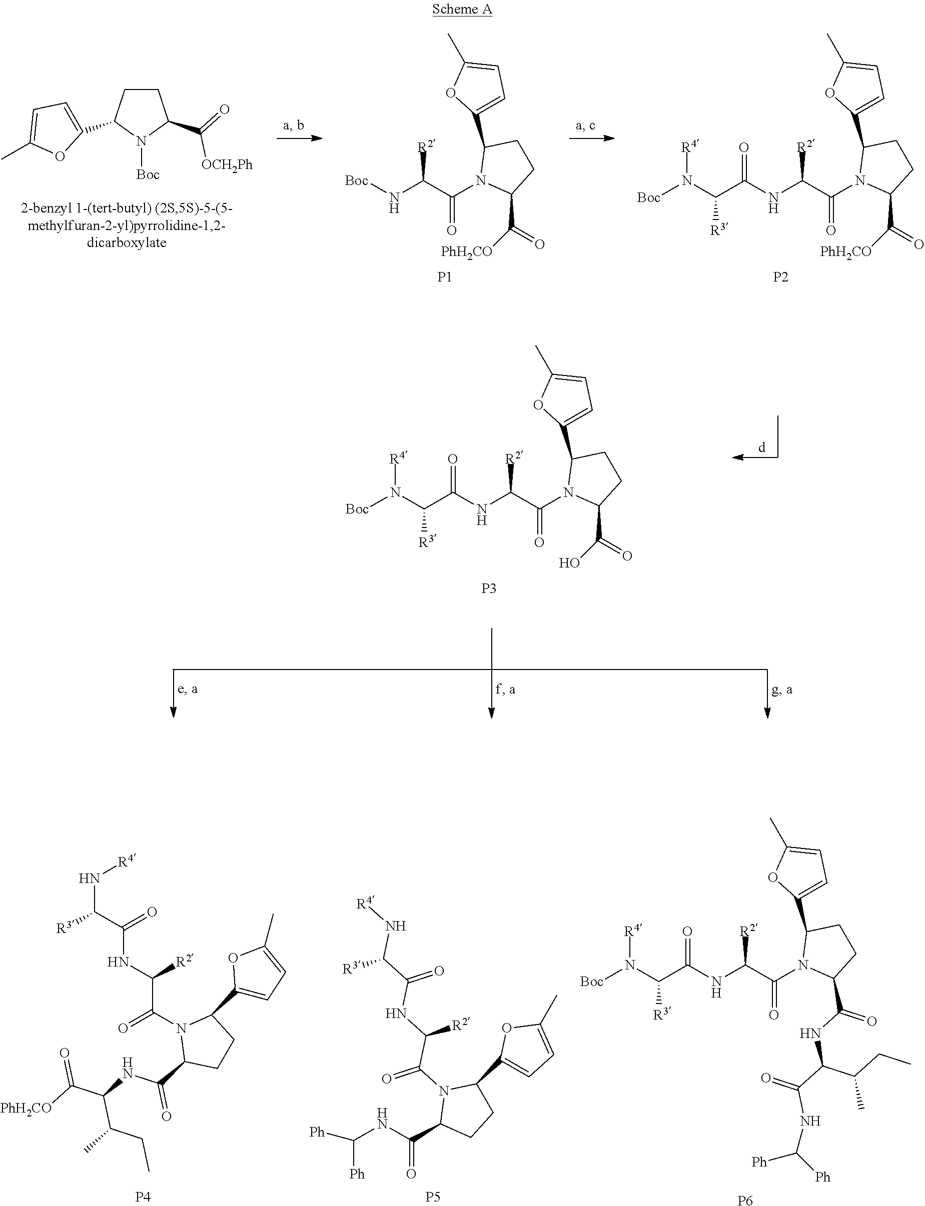

R², R³ and R⁴ are each independently selected from the group consisting of H, C₁-C₆ alkyl and C₄-C₈ cycloalkyl.

(Reagent and conditions: (a) 20% TFA/DCM; (b) BocNHCH(R^{2′})-OH, EDCI.HCl, HOBt, DIPEA, DCM/DMF (1:1); (c) BocN(R^{4′})CH(R^{3′})-OH, EDCI.HCl, HOBt, DIPEA, DCM/DMF (1:1); (d) 10 mol % Pd-C, H₂ by balloon, MeOH; (e) NH₂-Ile-OBn, EDCI.HCl, HOBt, DIPEA, DCM/DMF (1:1); (f) Benzhydrylamine, IBCF, NMM, THF, −15° C.); (g) NH₂-Ile-benzhydrylamide, HBTU, DIPEA, DMF)

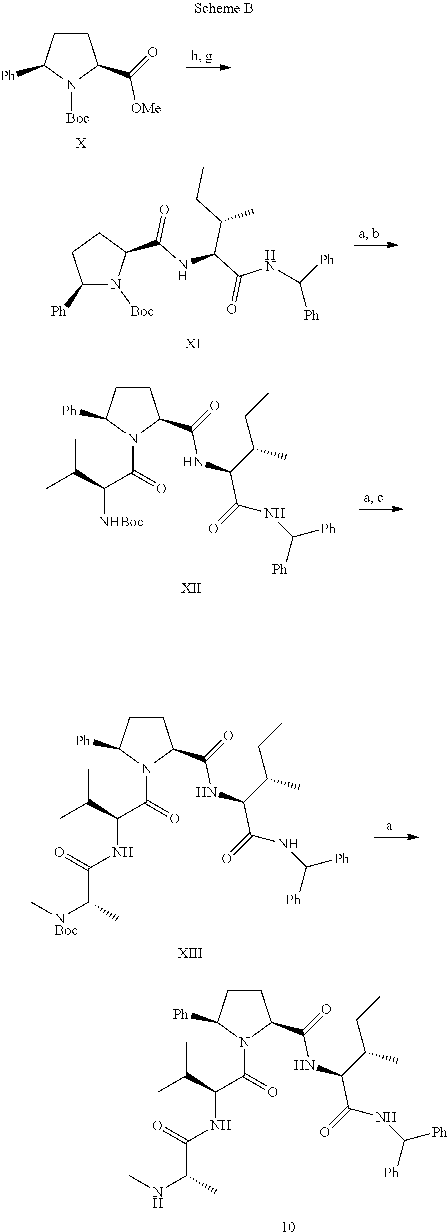

(Reagent and conditions: (a) 20% TFA/DCM; (b) Boc-Val-OH, EDCI.HCl, HOBt, DIPEA, DCM/DMF (1:1); (c) Boc-N-Me-Ala-OH EDCI.HCl, HOBt, DIPEA, DCM/DMF (1:1); (g) H-Ile-benzhydrylamide, HBTU, DIPEA, DMF h) LiOH, THF, MeOH, water;)

Based on the process described in Scheme A, the compounds 2-9; and based on process described in Scheme B, compound 10 are prepared in the following manner;
(i) Removing the Boc-group from the key intermediate I followed by coupling of the resulting amine with Boc-Val-OH to obtain a compound II (Scheme 1); removing Boc group of the compound-II and coupling the resulting amine with Boc-Ala-OH to obtain a compound III (Scheme 1) and catalytic hydrogenation of compound-III resulting in a free carboxylic acid IV which is coupled with either H-Ile-OBn or benzhydryl amine followed by acidolysis to yield the compounds 2 or 3 respectively (scheme 1).

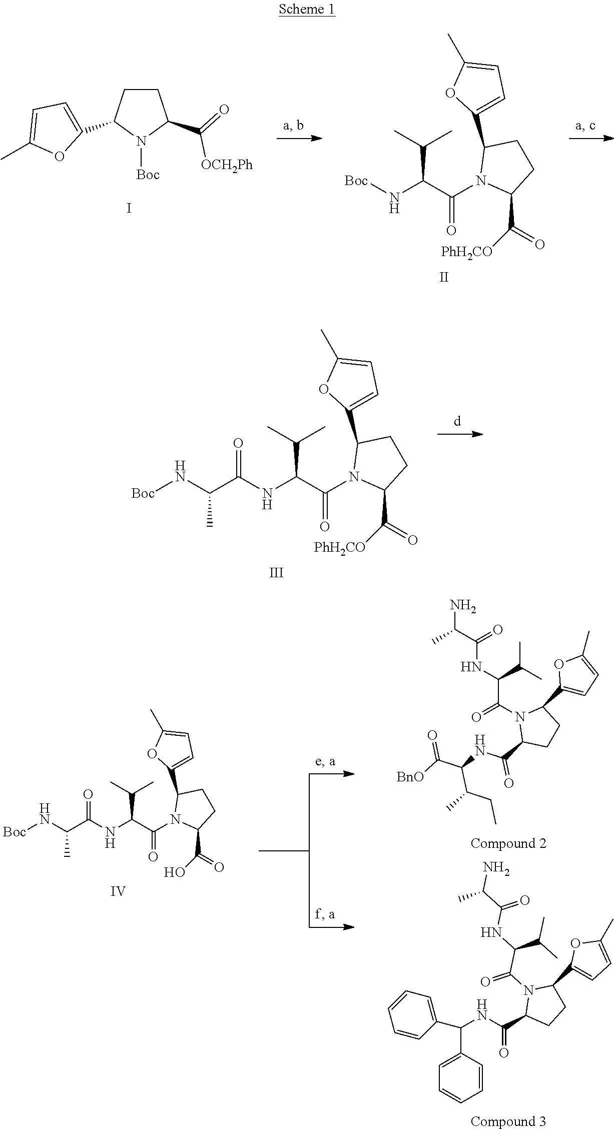

(Reagent and conditions:(a) 20% TFA/DCM; (b) Boc-Val-OH, EDCI.HCl, HOBt, DIPEA, DCM/DMF (1:1); (c) Boc-Ala-OH, EDCI.HCl, HOBt, DIPEA, DCM/DMF (1:1); (d) 10 mol % Pd-C, H2 by balloon; (e) H-Ile-OBn, EDCI.HC1, HOBt, DIPEA, DCM/DMF (1:1); (f) Benzhydrylamine, IBCF, NMM, THF, −15° C.)
(ii) Removing Boc group of a compound-II and coupling the resulting amine with Boc-N-Me-Ala-OH to obtain a compound V; catalytic hydrogenation of the compound V resulting in a compound VI with free carboxylic acid which is coupled with either H-Ile-OBn, benzhydryl amine or H-Ile-benzhydryl amide followed by acidolysis to yield the compounds 4, 5 or 6 respectively (scheme 2).

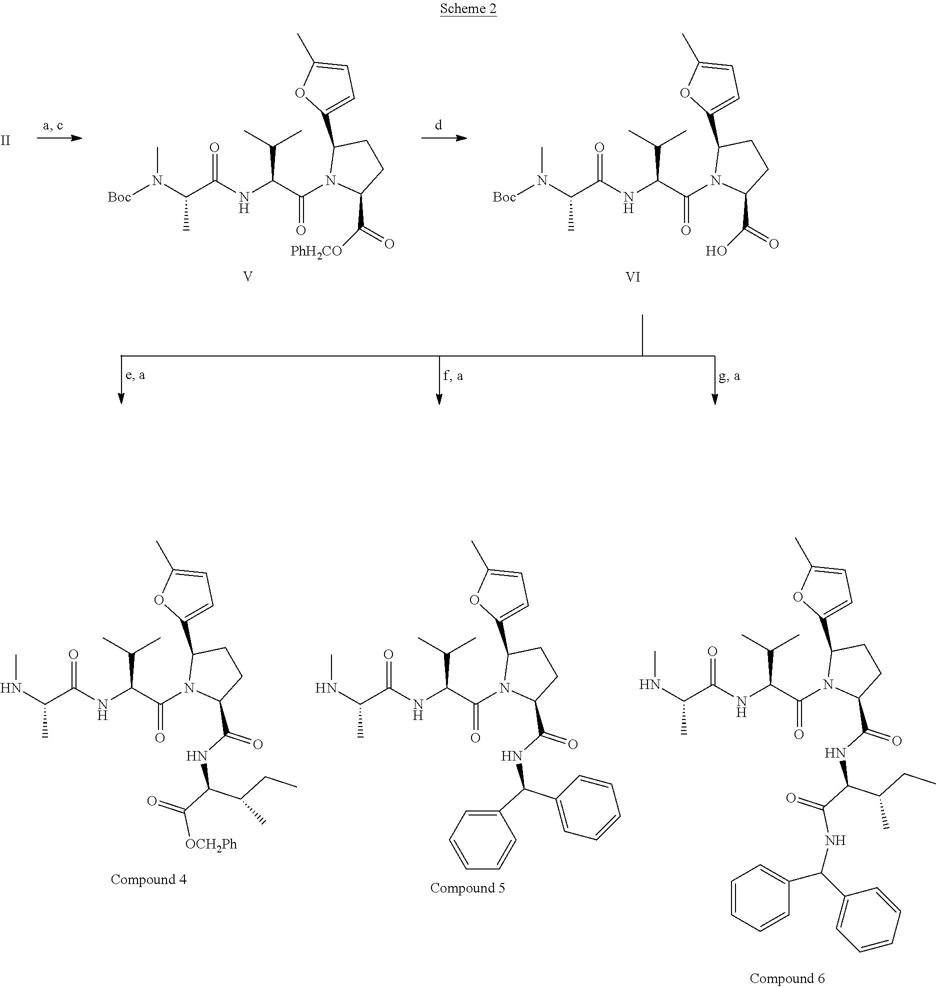

(Reagent and conditions: (a) 20% TFA/DCM; (b) Boc-Val-OH, HBTU, DIPEA, DMF;
(c) Boc-N-Me-Ala-OH, HBTU, DIPEA, DMF; (d) 10 mol % Pd-C, H₂ by balloon, MeOH;
(e) H-Ile-OBn, HBTU, DIPEA, DMF; (f) Benzhydrylamine, IBCF, NMM, THF, −15° C.;
(g) NH₂-Ile-benzhydrylamide, HBTU, DIPEA, DMF)
(iii) Removing Boc group of a Compound-I and coupling the resulting amine with Boc-Chg-OH to obtain a compound VII; acidolytic cleavage of the Boc- group of compound VII followed by coupling with Boc-N-Me-Ala-OH resulting in formation of a compound VIII and catalytic hydrogenation of compound VIII resulting in a compound IX which is coupled with either H-Ile-OBn, benzhydryl amine or Ile-benzhydryl amide followed by acidolysis to yield the compounds 7, 8 or 9 respectively (scheme 3).

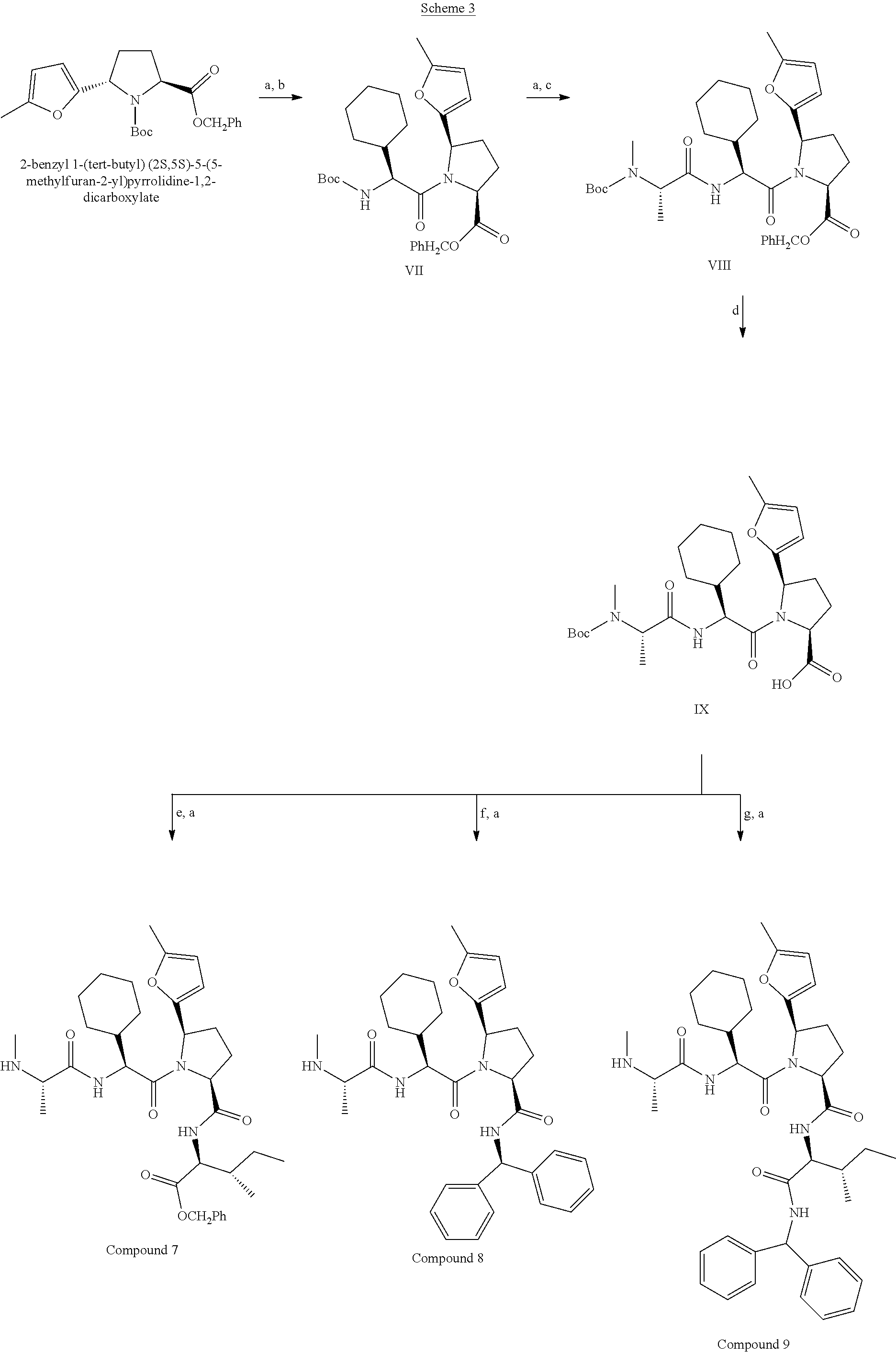

(Reagent and conditions: (a) 20% TFA/DCM; (b) Boc-Chg-OH, HBTU, DIPEA, DMF; (c) Boc-N(Me)-Ala-OH, HBTU, DIPEA, DMF; (d) 10 mol % Pd-C, H₂ by balloon, MeOH; (e) NH₂-Ile-OBn, HBTU, DIPEA, DMF; (f) Benzhydrylamine, IBCF, NMM, THF, −15° C.; (g) NH₂-Ile-benzhydrylamide, HBTU, DIPEA, DMF)
(iv) Saponification of compound X followed by coupling with H-Ile-benzhydryl amide to obtain a compound XI; Boc deprotection of compound XI followed by coupling with Boc-Val-OH to obtain compound XII; Boc deprotection of compound XII followed by coupling with Boc-N-Me-Ala-OH to obtain compound XIII; Boc deprotection of compound XIII to obtain compound 10 (scheme 4).

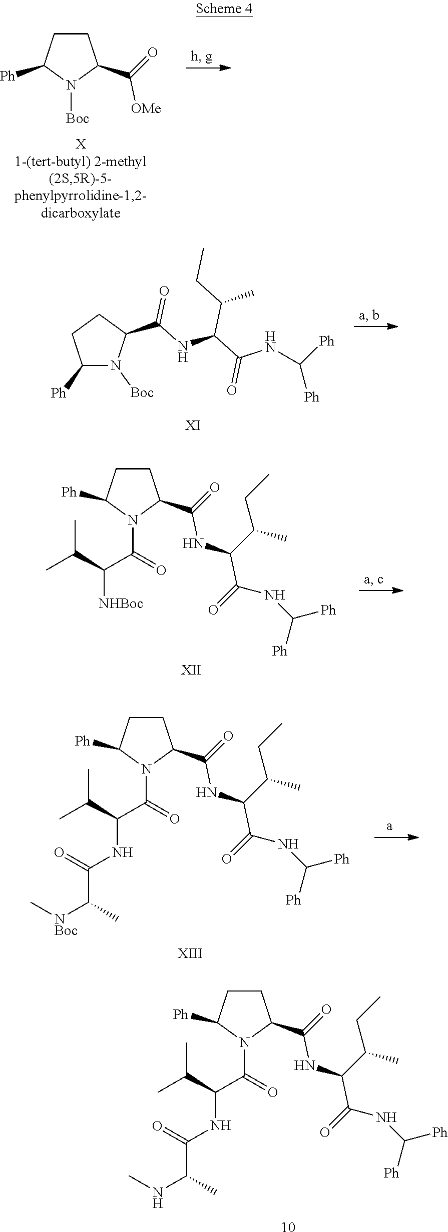

(Reagent and conditions: (h) LiOH, THF, Me0H, water; (g) H-Ile-benzhydrylamide, EDCI, HOBt, DIPEA, DCM; (a) 30% TFA/DCM; (b) Boc-Val-OH, EDCI, HOBt, DIPEA, DCM; (c) Boc-N-Me-Ala-OH, EDCI, HOBt, DIPEA, DCM;

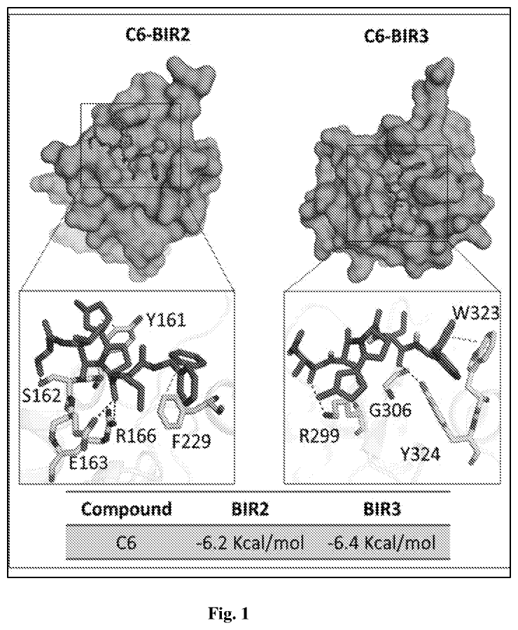

SMAC mimetic peptidomimetics of Formula-I of the present invention shows strong binding affinity to BIR-2 and BIR-3 domains of the XIAP. The binding affinity is measured by in-silico experiments (FIG. 1) as well as by protein binding assay using fluorescence polarization assay as shown in Table 1. After binding confirmation, the cytotoxic potential of the SMAC mimetic compounds was assessed against cancer cell lines versus non-human monkey kidney (VERO) cell line. All compounds showed cytotoxic activity against all cancer cells. Most importantly, out of total series of Smac mimetic compounds, Compound 6 shows potent cytotoxic activity against all cancer cells but has limited or minimal toxicity against non-human VERO cells proving its tumor cell selective nature (Table 1). Due to its tumor cell selective cytotoxic nature and potential binding characteristics, Compound 6 (C6) was selected as potent molecule for further experimental analysis.

TABLE 1
Binding and in-vitro cytotoxic efficacy of Smac mimetics

| | Target Binding | | | | | |
|---|---|---|---|---|---|---|
| | BIR2 | BIR3 | In vitro cytotoxicity at 10 μM dose | | | |
| Compound | Ki (μM) | Ki (μM) | SW 620 | HT29 | HCT 116 | VERO |
| 1 | 3.14 | 0.65 | 0.97 ± 2.3 | −0.01 ± 3.6 | −6.59 ± 6.6 | 5.74 ± 3.4 |
| 2 | >10 | >10 | 7.88 ± 8.0 | −1.83 ± 5.5 | −7.06 ± 6.9 | 24.24 ± 22.7 |
| 3 | 1.08 | 0.23 | 1.44 ± 2.1 | −0.46 ± 2.2 | −2.60 ± 6.1 | 3.89 ± 3.4 |
| 4 | 7.39 | 4.38 | −4.10 ± 0.0 | 11.99 ± 0.6 | −8.07 ± 6.6 | 4.54 ± 5.6 |
| 5 | 3.25 | 0.50 | 8.11 ± 11.3 | 40.88 ± 5.3 | 5.96 ± 1.2 | 11.39 ± 1.1 |
| 6 | 3.21 | 0.58 | 59.27 ± 0.1 | 65.03 ± 3.6 | 61.26 ± 2.9 | 18.41 ± 2.1 |
| 7 | >10 | 1.42 | 52.85 ± 22.4 | 71.60 ± 10.2 | 76.51 ± 2.3 | 63.17 ± 5.0 |
| 8 | 1.80 | 0.73 | 38.74 ± 12.6 | 66.04 ± 2.4 | 48.55 ± 6.7 | 39.40 ± 6.9 |
| 9 | >10 | 0.79 | 81.77 ± 3.3 | 72.69 ± 6.9 | 66.15 ± 15.0 | 59.43 ± 11.7 |

Further, the IC₅₀ concentration of C6 against diverse cancer cell lines was determined and it was observed that the SMAC mimetic peptidomimetics of Formula-I shows potent activity against various cancer cell lines including but not limited to colon, breast, kidney, prostate, brain, ovary, pancreas, liver, melanoma, leukemia and lymphoma. (Table 2).

TABLE 2
Cytotoxic activity of C6 against various cells

| Cancer Type | Cell Line | IC₅₀ (μM) |
|---|---|---|
| Colon | HT29 | 6.66 |
| Colon | SW620 | 4.31 |
| Colon | HCT116 | 8.93 |
| Colon (Mouse) | CT26 | >10 |
| Breast | MDAMB231 | 4.15 |
| Breast | HCC1806 | 5.79 |
| Breast | MCF-7 | 5.02 |
| Breast (Mouse) | 4T1 | 4.17 |
| Lung | A-549 | >10 |
| Kidney | 786-O | 7.2 |
| Prostrate | PC-3 | 6.02 |
| Brain | A172 | 6.88 |
| Ovary | SK-OV-3 | 3.91 |
| Pancreas | PANC-1 | 5.83 |
| Liver | HEP-G2 | 7.3 |
| Melanoma (Mouse) | B-16 | 6.7 |
| Leukemia | K562 | 9.10 |
| Lymphoma | U937 | 8.98 |
| Kidney (monkey) | VERO | >10 |

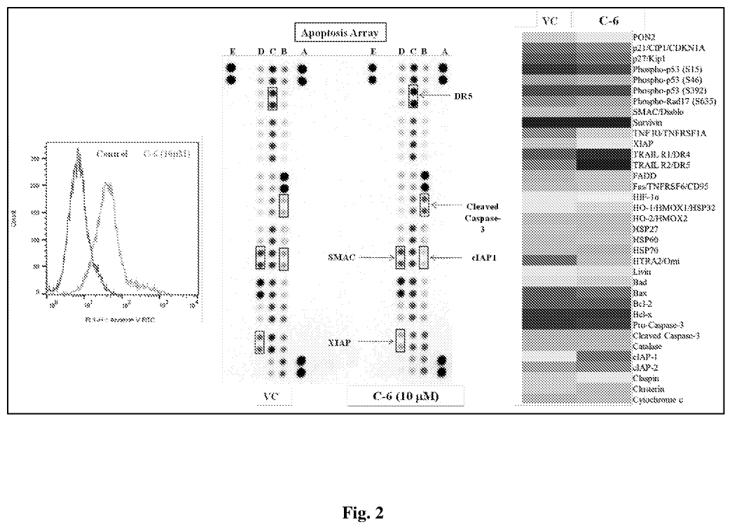

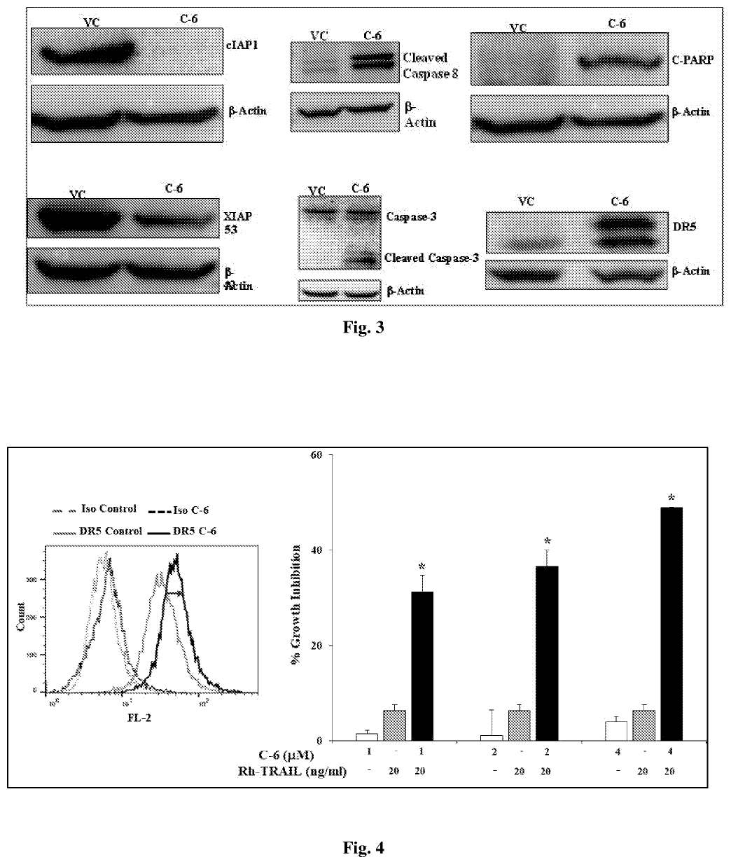

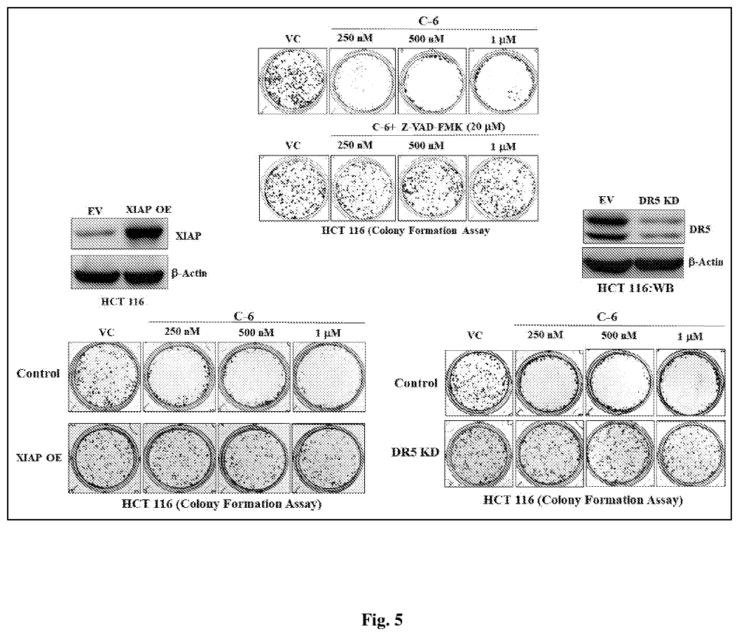

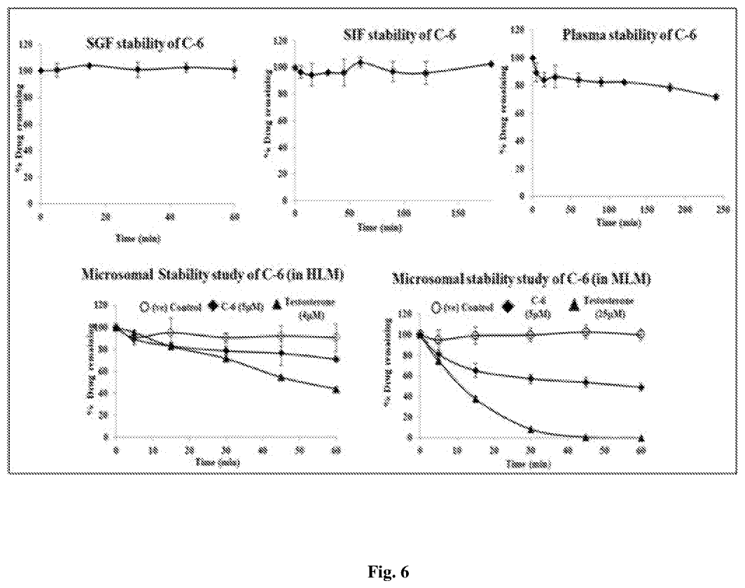

The SMAC mimetic C6 promotes cell death in cancer cells by turning on hallmark SMAC driven apoptotic features like cleavage of caspases and degradation of cIAP1 etc. as shown in FIG. 2 and FIG. 3. Further, its apoptotic functions are highly dependent on target engagement and it promotes apoptosis in TRAIL resistant cells as shown in FIG. 4 and FIG. 5. Stability and pharmacokinetic analysis of C6 shows that it is highly stable in SIF, SGF, microsome (FIG. 6) and have significant bioavailability via Subcutaneous and Oral routes of administration as shown in Table-3 suggesting druggable potential of the SMAC mimetics disclosed in the present invention.

TABLE 3
Pharmacokinetic properties of C6

| Parameter (Unit) | IV route | SC route | Oral route |
|---|---|---|---|
| Cₘₐₓ (ng/mL) | 10215 ± 2633.37 | 5018.33 ± 853.66 | 4490.00 ± 1514.05 |
| AUC_{0-∞} (h * ng/mL) | 4107.24 ± 852.34 | 17439.13 ± 2220.28 | 17227.95 ± 3433.39 |
| Tₘₐₓ (h) | 0.08 | 0.88 ± 0.21 | 1.08 ± 0.47 |
| T_{1/2} (h) | 0.19 ± 0.05 | 2.10 ± 0.15 | 3.84 ± 1.80 |
| C1 (L/hr/Kg) | 1.02 ± 0.24 | 1.75 ± 0.24 | 1.79 ± 0.32 |
| Vd (L/Kg) | 0.28 ± 0.12 | 5.32 ± 0.98 | 9.52 ± 3.75 |
| MRT (h) | 1.72 ± 0.46 | 8.92 ± 1.35 | 4.18 ± 1.81 |
| Absolute | — | 56.61 ± 7.21 | 55.93 ± 11.15 |
| Bioavailability (%) | | | |

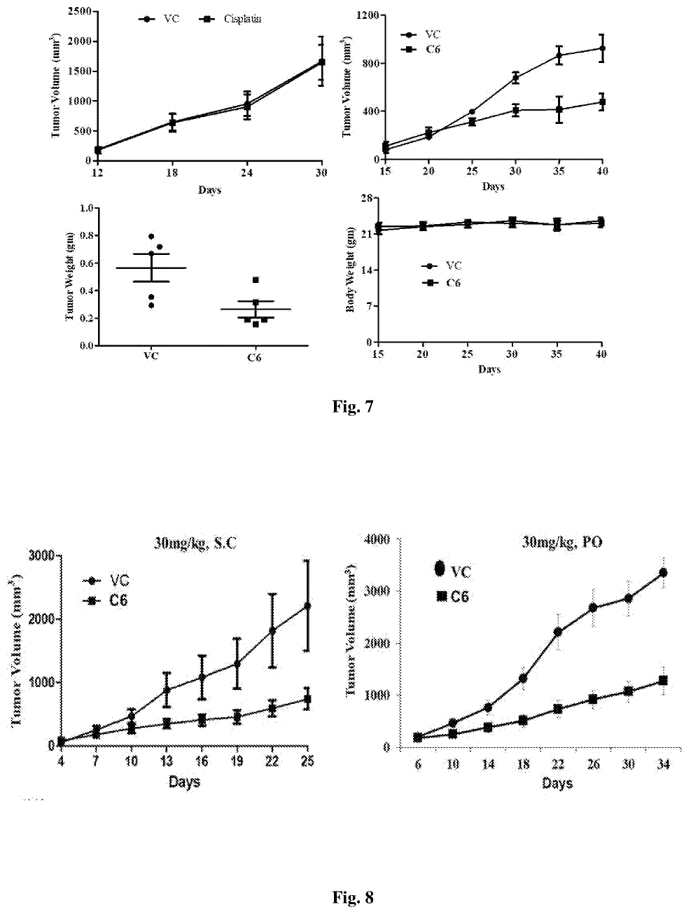

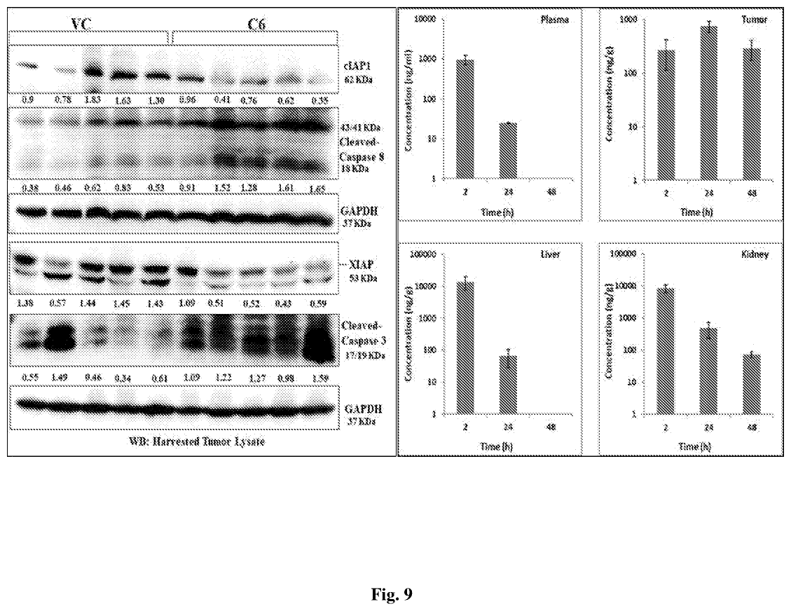

C6 is showing robust in-vivo anti-tumor activity by intraperitoneal, sub-cutaneous and oral route of administration as shown in FIG. 7 and FIG. 8. It is also in vivo active against cisplatin resistant colon cancer. C6 is found to be well tolerated and non-toxic at the dose where no weight loss was observed in animals during the course of treatment as shown in FIG. 7 and FIG. 8. Further, C6 is reaching to the tumor site through oral route of administration and engaging its target like XIAP/IAP degradation and cleavage of caspases (FIG. 9).

### EXAMPLES

Following examples are given to support, but not limited, to the present invention.

### Example 1

### Synthesis of benzyl ((2S,5R)-1-(L-alanyl-L-valyl)-5-(5-methylfuran-2-yl)pyrrolidine-2-carbonyl)-L-isoleucinate as referred in Scheme 1 (compound 2)

(2S,5S)-2-benzyl 1-tert-butyl 5-(5-methylfuran-2-yl)pyrrolidine-1,2-dicarboxylate (850 mg, 2.2 mmol) was stirred in 20% TFA/DCM for 1 h. After that, the reaction mixture was concentrated to dryness under reduce pressure. This amine was then dissolved in anhydrous DMF (2 mL) and was added to the prestirred solution of Boc-Val-OH (956 mg, 4.4 mmol) and HBTU (1.7 gm, 4.4 mmol) in dry DMF (3 mL) at 0° C. under nitrogen atmosphere followed by addition of DIPEA (1.2 mL, 6.6 mmol). The reaction mixture was further stirred for additional 4-6 h at room temperature. After completion of reaction, water (30-40 mL) was added. Aqueous solution was extracted with ethyl acetate (3×60 mL). The combined organic layer was washed with 10% citric acid (aq.), 10% NaHCO₃ (aq.) and finally with brine. The organic layer was dried with anhydrous sodium sulphate and the solvent was removed under reduced pressure to obtain the crude product, which was purified by column chromatography using 18% ethyl acetate/hexane as eluent to give the intermediate compound (2S ,5R)-benzyl 1-((S)-2-(tert-butoxycarbonylamino)-3-methylbutanoyl)-5-(5-methylfuran-2-yl)pyrrolidine-2-carboxylate (II) as brownish gummy oil in 56.8% yield (605.6 mg, 1.25 mmol HRMS (ESI) (M+H)⁺ calculated for C₂₇H₃₇N₂O₆⁺=485.2646, found 485.2645.

The intermediate compound II (484.6 mg, 1 mmol) was stirred in 20% TFA/DCM for 1 h. After that, reaction mixture was concentrated to dryness under reduce pressure. This amine was then dissolved in anhydrous DMF (2 mL) and was added to the pre-stirred solution of Boc-Ala-OH (378.4 mg, 2 mmol) and HBTU (758.5 mg, 2 mmol) in dry DMF (3 mL) at 0° C. under nitrogen atmosphere followed by addition of DIPEA (0.56 mL, 3 mmol). The reaction mixture was further stirred for additional 4-6 h at room temperature.

After completion of reaction and usual work up, the intermediate compound (2S,5R)-benzyl 1-((S)-2-((S)-2-(tert-butoxyc arbonylamino)prop anamido)-3 -methylbutanoyl)-5 -(5-methylfuran-2-yl)pyrrolidine-2-carboxylate (III) was obtained as yellowish gummy oil in 65% yield (361 mg, 0.65 mmol). HRMS (ESI) (M+H)⁺ calculated for C₃₀H₄₂N₃O₇⁺=556.3017, found 556.3022.

To a stirred solution of compound III (1 mmol) in methanol (5 mL) was added 10 mol % Pd-C (0.1 mmol) and subjected to hydrogenation by purging hydrogen gas through balloon for 30 minutes. After that Pd was filtered using celite pad and filterate was concentrated in vacuo to give the free acid (2S,5R)-1-((S)-2-((S)-2-(tert-butoxycarbonylamino)propanamido)-3-methylbutanoyl)-5-(5-methylfuran-2-yl)pyrrolidine-2-carboxylic acid (IV) which was used in the next steps without further purification. After that isoleucine benzyl ester (13.3 mg, 0.06 mmol) was dissolved in anhydrous DMF (1 mL) and was added to the pre-stirred solution of compound IV (28 mg, 0.06 mmol) and HBTU (22.7 mg, 0.06 mmol) in dry DMF (1 mL) at 0° C. under nitrogen atmosphere followed by addition of DIPEA (0.034 mL, 0.18 mmol). The reaction mixture was further stirred for additional 4-6 h at room temperature. After completion of reaction, and usual work up, the protected tetrapeptide (2S,3S)-benzyl 2-((2S,5R)-1-((S)-2-((S)-2-(tert-butoxycarbonylamino)propanamido)-3-methylbutanoyl)-5-(5-methylfuran-2-yl)pyrrolidine-2-carboxamido)-3-methylpentanoate was obtained as yellowish gummy oil in 86% yield (34.8 mg, 0.05 mmol). HRMS (ESI) (M+H)⁺ calculated for C₃₆H₅₃N₄O₈⁺=669.3858, found 669.3865.

Protected peptide (2S,3S)-benzyl 2-((2S,5R)-1-((S)-2-((S)-2-(tert-butoxycarbonylamino)propanamido)-3-methylbutanoyl)-5-(5-methylfuran-2-yl)pyrrolidine-2-carboxamido)-3-methylpentanoate (67 mg, 0.1 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired peptide compound (2S,3S)-benzyl 2(2S,5R)-1(S)-2(S)-2-aminopropanamido)-3-methylbutanoyl)-5-(5-methylfuran-2-yl)pyrrolidine-2-carboxamido)-3-methylpentanoate (2) as a white powder in 65% yield (37 mg, 0.065 mmol). The compound 2 was found to be 97% pure at 220 nm on analytical RP-HPLC. ¹H NMR (500 MHz, DMSO-d₆): δ/ppm=8.67 (d, 1H, J=8.2 Hz), 8.08 (br, 2H), 7.93 (d, 1H, J=8.2 Hz), 7.38-7.31 (m, 5H), 6.53 (br, 1H, J=3.1 Hz), 6.04 (brdd, 1H, J=1.0, 3.1 Hz), 5.41 (m, 1H), 5.13 (m, 2H), 4.41-4.22 (m, 3H), 3.88 (br, 1H), 2.24 (s, 3H), 2.12-1.76 (m, 6H), 1.42-1.34 (m, 1H), 1.31 (d, 3H, J=7.0 Hz), 1.23-1.12 (m, 1H), 0.86-0.77 (m, 9H), 0.61 (d, 3H, J=6.7 Hz); HRMS (ESI) (M+H)⁺ calculated for C₃₁H₄₅N₄O₃⁺=569.3334, found 569.3335.

### Example 2

### Synthesis of (2S,5R)-1-(L-alanyl-L-valyl)-N-benzhydryl-5-(5-methylfuran-2-yl)pyrrolidine-2-carboxamide as referred in scheme 1 (Compound 3)

To the solution of compound IV (50 mg, 0.12 mmol) in tetrahydrofuran (1 mL) at —15° C., was added N-methyl morpholine (17.5 μL, 0.16 mmol, 1.5 equiv). After five minutes, isobutylchloroformate (17.5 μL, 0.144 mmol, 1.2 equiv) was added to the reaction mixture. Then after five minutes, benzhydrylamine (19 μL, 0.144 mmol, 1.2 equiv) was added and the reaction mixture was stirred for additional 1 h at −15° C. After completion of reaction and usual work up, compound protected peptide tert-butyl (S)-1-((S)-1-((2S,5R)-2-(benzhydrylcarbamoyl)-5-(5-methylfuran-2-yl)pyrrolidin-1-yl)-3-methyl-1-oxobutan-2-ylamino)-1-oxopropan-2-ylcarbamate (Boc-Ala-Val-Fro-bezhydrylamide) in 71% yield (53 mg, 0.085 mmol HRMS (ESI) (M+H)⁺ calculated for C₃₆H₄₇N₄O₆⁺=631.3490, found 631.3485.

Compound tert-butyl (S)-1-((S)-1-((2S,5R)-2-(benzhydrylcarbamoyl)-5-(5-methylfuran-2-yl)pyrrolidin-1-yl)-3-methyl-1-oxobutan-2-ylamino)-1-oxopropan- 2-ylcarbamate (Boc-Ala-Val-Fro-bezhydrylamide) (53 mg, 0.085 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired peptide (2S,5R)-1-((S)-2-((S)-2-aminoprop anamido)-3-methylbutanoyl)-N-benzhydryl-5-(5-methylfuran-2-yl)pyrrolidine-2-carboxamide 3) as a white powder in 70% yield (32 mg, 0.06 mmol). The compound 3 was found to be 96% pure at 220 nm on analytical RP-HPLC. ¹H NMR (500 MHz, DMSO-d₆): δ/ppm=8.69 (d, 1H, J=8.2 Hz), 8.42 (d, 1H, J=8.3 Hz), 8.08 (br d, 2H, J=3.7 Hz), 7.35-7.21 (m, 10H), 6.52 (d, 1H, J=2.9 Hz), 6.09 (d, 1H, J=8.2 Hz), 6.0 (br d, 1H, J=2.9 Hz), 5.44 (br d, 1H, J=7.4 Hz), 4.45 (m, 1H), 4.22 (m, 1H), 3.87 (m, 1H), 2.20-2.14 (m, 1H), 2.15 (s, 3H), 2.10-1.98 (m, 2H), 1.96-1.89 (m, 1H), 1.88-1.80 (m, 1H), 1.30 (d, 3H, J=6.9 Hz), 0.77 (d, 3H, J=6.7 Hz), 0.55 (d, 3H, J=6.7 Hz); HRMS (ESI) (M+H)⁺ calculated for C₃₁H₃₉N₄O₄⁺=531.2966, found 531.2958.

### Example 3

Synthesis of (2S,3S)-benzyl 3-methyl-2-((2S,,5R)-1(S)-3-methyl-2-(S)-2-(methylamino)propanamido)butanoyl)-5-(5-methylfuran-2-yl)pyrrolidine-2-carboxamido)pentanoate as referred in Scheme 2 (Compound 4)

The intermediate compound II (obtained in Scheme 1) (484.6 mg, 1 mmol) was stirred in 20% TFA/DCM for 1 h. After that reaction mixture was concentrated to dryness under reduce pressure. This amine was then dissolved in anhydrous DMF (2 mL) and was added to the pre-stirred solution of Boc-N-Me-Ala-OH (406 mg, 2 mmol) and HBTU (758.5 mg, 2 mmol) in dry DMF (3 mL) at 0° C. under nitrogen atmosphere followed by addition of DIPEA (0.56 mL, 3 mmol). The reaction mixture was further stirred for additional 4-6 h at room temperature. After completion of reaction and usual work up, the compound (2S,5R)-benzyl 1-((S)-2-((S)-2-(tert-butoxycarbonyl(methyl)amino)propanamido)-3-methylbutanoyl)-5-(5-methylfuran-2-yl)pyrrolidine-2-carboxylate (V) was obtained as brownish gummy oil in 62% yield (353 mg, 0.62 mmol). HRMS (ESI) (M+H)⁺ calculated for C₃₁H₄₄N₃O₇⁺=570.3174, found 570.3407.

To a stirred solution of compound V (570 mg, 1 mmol) in methanol/tetrahydrofuran (5 mL, 1:1) was added 10 mol % Pd-C (0.1 mmol) and subjected to hydrogenation by purging hydrogen gas through balloon for 30 minutes. After that Pd was filtered using celite pad and filterate was concentrated in vacuo to give the free acid (2S,5R)-1-((S)-2-((S)-2-(tert-butoxycarbonyl(methyl)amino)propanamido)-3-methylbutanoyl)-5-(5-methylfuran-2-yl)pyrrolidine-2-carboxylic acid (VI) which was used in the next steps without further purification.

The isoleucine benzyl ester (55.5 mg, 0.25 mmol) was dissolved in anhydrous DMF (1 mL) and was added to the pre-stirred solution of compound VI (120 mg, 0.25 mmol) and HBTU (95 mg, 0.25 mmol) in dry DMF (2 mL) at 0° C. under nitrogen atmosphere followed by addition of DIPEA (0.13 mL, 0.75 mmol). After completion of reaction and usual work up the crude product, which was purified by column chromatography using 5% methanol/dichloromethane as eluent to give the compound Boc protected tetra peptide (2S,3S)-benzyl 2-((2S,5R)-1-((S)-2-((S)-2-(tert-butoxycarbonyl(methyl)amino)propanamido)-3-methylbutanoyl)-5-(5-methylfuran-2-yl)pyrrolidine-2-carboxamido)-3-methylpentanoate (Boc-N(Me)-Ala-Val-Fro-Ile-OBn) as brownish gummy oil in 80% yield (136 mg, 0.2 mmol). HRMS (ESI) (M+H)⁺ calculated for C₃₇H₅₅N₄O₈⁺=683.4014, found 683.4011.

Compound (2S 3S)-benzyl 2-((2S,5R)-1-((S)-2-((S)-2-(tert-butoxycarbonyl(methyl)amino)propanamido)-3-methylbutano yl)-5-(5-methylfuran-2-yl)pyrrolidine-2-carboxamido)-3-methylpentanoate (53.5 mg, 0.08 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired compound (2S,3S)-benzyl 3-methyl-2((2S,5R)-1-((S)-3-methyl-2-((S)-2-(methylamino)propanamido)butanoyl)-5-(5-methylfuran-2-yl)pyrrolidine-2-carboxamido)pentanoate (4) as a white powder. Yield (28 mg, 62%). The compound 4 was found to be 99% pure at 220 nm on analytical RP-HPLC.¹H NMR (500 MHz, DMSO-d₆): δ/ppm=8.96 (br, 1H), 8.85 (d, 1H, J=8.7 Hz), 7.93 (d, 1H, J=8.5 Hz), 7.38-7.30 (m, 5H), 6.54 (br d, 1H, J=3.2 Hz), 6.03 (brdd, 1H, J =1.0, 3.2 Hz), 5.36 (br d, 1H, J=7.7 Hz), 5.16-5.08 (m, 2H), 4.42-4.24 (m, 3H), 3.82 (br, 1H), 2.51 (s, 3H), 2.23 (s, 3H), 2.11-1.94 (m, 3H), 1.92-1.75 (m, 3H), 1.43-1.34 (m,1H), 1.33 (d, 3H, J=7.1 Hz), 1.22-1.13 (m,1H), 0.86-0.76 (m, 9H), 0.62 (d, 3H, J=6.9 Hz); HRMS (ESI) (M+H)⁺ calculated for C₃₂H₄₇N₄O₆⁺=583.3490, found 583.3482.

### Example 4

### Synthesis of (2S,5R)-N-benzhydryl-1-(methyl-L-alanyl-L-valyl)-5-(5-methylfuran-2-yl)pyrrolidine-2-carboxamide as referred in Scheme 2 (Compound 5)

To the solution of intermediate compound VI (58 mg, 0.12 mmol) in tetrahydrofuran (1 mL) at −15° C., was added N-methyl morpholine (17.5 μL, 0.16 mmol, 1.5 equiv). After five minutes isobutylchloroformate (17.5 μL, 0.144 mmol, 1.2 equiv) was added to the reaction mixture. Then after five minutes, benzhydrylamine (19 μL, 0.144 mmol, 1.2 equiv) was added and the reaction mixture was stirred for additional 1 h at −15° C. After completion of reaction and usual work up the crude product was obtained, which was purified by 4% methanol/dichloromethane to give compound Boc protected peptide tert-butyl (S)-1-((S)-1-((2S,5R)-2-(benzhydrylcarbamoyl)-5-(5-methylfuran-2-yl)pyrrolidin-1-yl)-3-methyl-1-oxobutan-2-ylamino)-1-oxopropan-2-yl(methyl)carbamate (Boc-N(Me)-Ala-Val-Fro-benzhydrylamide) in 75% yield (57 mg, 0.09 mmol). HRMS (ESI) (M+H)⁺ calculated for C₃₇H₄₉N₄O₆⁺=645.3647, found 645.3646.

Compound tert-butyl (S)-1-((S)-1-((2S,5R)-2-(benzhydrylcarbamoyl)-5-(5-methylfuran-2-yl)pyrrolidin-1-yl)-3-methyl-1-oxobutan-2-ylamino)-1-oxopropan-2-yl(methyl)carbamate (Boc-N(Me)-Ala-Val-Fro-benzhydrylamide) (57 mg, 0.08 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired compound (2S,5R)-N-benzhydryl-1-((S)-3-methyl-2-((S)-2-(methylamino)propanamido)butanoyl)-5-(5-methylfuran-2-yl)pyrrolidine-2-carboxamide (5) as a white powder in 68% yield (30 mg, 0.055 mmol).

The compound 5 was found to be more than 99% pure at 220 nm on analytical RP-HPLC. ¹H NMR (500 MHz, DMSO-d₆): δ/ppm=8.87 (br, 1H), 8.85 (d, 1H, J=8.9 Hz), 8.41 (d, 1H, J=8.2 Hz), 7.35-7.21 (m, 10H), 6.54 (br d, 1H, J=3.0 Hz), 6.10 (d, 1H, J =8.5 Hz), 5.99 (brdd, 1H, J =1.0, 3.0 Hz), 5.40 (br d, 1H, J =6.1 Hz), 4.47-4.43 (m, 1H), 4.27 (t, 1H, J=8.7 Hz), 3.83 (m, 1H), 2.51 (s, 3H), 2.21-2.13 (m,1H), 2.15 (s, 3H), 2.12-1.81 (m, 4H), 1.34 (d, 3H, J=6.9 Hz), 0.77 (d, 3H, J=6.7 Hz), 0.57 (d, 3H, J=6.7 Hz); HRMS (ESI) (M+H)⁺ calculated for C₃₂H₄₁iN₄O₄⁺545.3122, found 545.3116.

### Example 5

### Synthesis of (2S,5R)-N-4S,3S)-1-(benzhydrylamino)-3-methyl-1-oxopentan-2-yl)-1-(methyl-L-alanyl-L-valyl)-5-(5-methylfuran-2-yl)pyrrolidine-2- carboxamide as referred in Scheme 2 (compound 6)

The isoleucine benzhdrylamide (178 mg, 0.6 mmol) was dissolved in anhydrous DMF (1 mL) and was added to the pre-stirred solution of compound VI (288 mg, 0.6 mmol) and HBTU (227.5 mg, 0.6 mmol) in dry DMF (3 mL) at 0° C. under nitrogen atmosphere followed by addition of DIPEA (0.34 mL, 1.8 mmol). The reaction mixture was further stirred for additional 4-6 h at room temperature. After completion of reaction and usual work up, the crude product thus obtained was purified by column chromatography using 5% methanol/dichloromethane as eluent to give the compound Boc protected tert-butyl (S)-1-((S)-1-((2S,5R)-2-((2S ,3S)-1-(benzhydrylamino)-3 -methyl-l-oxopentan-2-ylcarbamo y1)-5-(5-methylfuran-2-yl)pyrrolidin-1-yl)-3-methyl-1-oxobutan-2-ylamino)-1-oxopropan-2-yl(methyl)carbamate as brownish gummy oil in 83% yield (379 mg, 0.5 mmol). HRMS (ESI) (M+H)⁺ calculated for C₄₃H₆₀N₅O₇=758.4487, found 758.4483.

Compound tert-butyl (S)-1-((S)-1(2S,5R)-24(2S ,3S)-1-(benzhydrylamino)-3-methyl-1-oxopentan-2-ylcarbamoyl)-5-(5-methylfuran-2-yl)pyrrolidin-1-yl)-3-methyl-1-oxobutan-2-ylamino)-1-oxopropan-2-yl(methyl)carbamate (250 mg, 0.32 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired Compound (2S,5R)-N-((2S,3S)-1-(benzhydrylamino)-3-methyl-1-oxopentan-2-yl)-1-((S)-3-methyl-2-((S)-2-(methylamino)propanamido)butanoyl)-5-(5-methylfuran-2-yl)pyrrolidine-2-carboxamide (6) as a white powder in 56% yield (119 mg, 0.18 mmol). The compound 6 was found to be 99% pure at 220 nm on analytical RP-HPLC. ¹H NMR (500 MHz, DMSO-d₆): δ/ppm=8.93 (d, 1H, J=8.8 Hz), 8.86 (br, 1H), 8.83 (d, 1H, J=8.4 Hz), 7.56 (d, 1H, J=8.9 Hz), 7.33-7.21 (m, 10H), 6.52 (br d, 1H, J=3.0 Hz), 6.11 (d, 1H, J=8.7 Hz), 6.03 (br d, 1H, J=2.5 Hz), 5.35 (m, 1H), 4.40-4.30 (m, 3H), 3.8 (br, 1H), 2.50 (s, 3H), 2.25 (s, 3H), 2.13-1.68 (m, 6H), 1.45-1.37 (m, 1H), 1.33 (d, 3H, J=6.9 Hz), 1.09-0.98 (m, 1H), 0.80-0.74 (m, 9H), 0.59 (d, 3H, J=6.7 Hz); HRMS (ESI) (M+H)⁺ calculated for C₃₈H₅₂N₅O₅=658.3963, found 658.3953.

### Example 6

### Synthesis of (2S,3S)-benzyl 2-((2S,5R)-1-((S)-2-cyclohexyl-2-((S)-2-(methylamino)propanamido)acetyl)-5-(5-methylfuran-2-yl)pyrrolidine-2-carboxamido)-3-methylpentanoate as referred in Scheme 3 (Compound 7)

2-benzyl 1-(tert-butyl) (2S,5S)-5-(5-methylfuran-2-yl)pyrrolidine-1,2-dicarboxylate (850 mg, 2.2 mmol) was stirred in 20% TFA/DCM for 1 h. After that, the reaction mixture was concentrated to dryness under reduce pressure. This amine was then dissolved in anhydrous DMF (2 mL) and was added to the prestirred solution of Boc-Chg-OH (1.13 gm, 4.4 mmol) and HBTU (1.7 gm, 4.4 mmol) in dry DMF (3 mL) at 0° C. under nitrogen atmosphere followed by addition of DIPEA (1.2 mL, 6.6 mmol). The reaction mixture was further stirred for additional 4-6 h at room temperature After completion of reaction and usual work up and purification, the intermediate compound (2S,5R)-benzyl 1-((S)-2-(tert-butoxycarbonylamino)-2-cyclohexylacetyl)-5-(5-methylfuran-2-yl)pyrrolidine-2-carboxylate (VII) as yellowish gummy oil in 54.5% yield (629 mg, 1.2 mmol). HRMS (ESI) (M+H)⁺ calculated for C₃₀H₄₁N₂O₆⁺=525.2959, found 525.2952.

The compound VII (524.6 mg, 1 mmol) were stirred in 20% TFA/DCM for 1 h. After that reaction mixture was concentrated to dryness under reduce pressure. This amine was then dissolved in anhydrous DMF (2 mL) and was added to the prestirred solution of N-Boc-N-methylalanine (406 mg, 2 mmol) and HBTU (758.5 mg, 2 mmol) in dry DMF (3 mL) at 0° C. under nitrogen atmosphere followed by addition of DIPEA (0.56 mL, 3 mmol). The reaction mixture was further stirred for additional 4-6 h at room temperature. After completion of reaction water (10-20 mL) was added. Aqueous solution was extracted with ethyl acetate (3×30 mL). The combined organic layer was washed with 10% citric acid (aq.), 10% NaHCO₃ (aq.) and finally with brine. The organic layer was dried with anhydrous sodium sulphate and the solvent was removed under reduced pressure to obtain the crude product, which was purified by column chromatography using 32% ethyl acetate/hexane as eluent to give the intermediate compound (2S,5R)-benzyl 1-((S)-2-((S)-2-(tert-butoxycarbonyl(methyl)amino)propanamido)-2-cyclohexylacetyl)-5-(5-methylfuran-2-yl)pyrrolidine-2-carboxylate (VIII) as brownish gummy oil in 62% yield (378 mg, 0.62 mmol). HRMS (ESI) (M+H)⁺ calculated for C₃₄H₄₈N₃O₇⁺=610.3487, found 610.3378.

To a stirred solution of compound VIII (378 mg, 0.62 mmol) in methanol/tetrahydrofuran (5 mL, 1:1) was added 10 mol % Pd-C (0.06 mmol) and subjected to hydrogenation by purging hydrogen gas through balloon for 30 minutes. After that Pd was filtered using celite pad and the filtrate was concentrated in vacuo to give free acid (2S,5R)-1-((S)-2-((S)-2-(tert-butoxycarbonyl(methyl)amino)propanamido)-2-cyclohexylacetyl)-5-(5-methylfuran-2-yl)pyrrolidine-2-carboxylic acid (IX) which was used in the next steps without further purification. The isoleucine benzyl ester (55.5 mg, 0.25 mmol) was dissolved in anhydrous DMF (1 mL) and was added to the pre-stirred solution of Boc-N(Me)-Ala-Chg-Fro-OH (130 mg, 0.25 mmol) and HBTU (95 mg, 0.25 mmol) in dry DMF (2 mL) at 0° C. under nitrogen atmosphere followed by addition of DIPEA (0.13 mL, 0.75 mmol). The reaction mixture was further stirred for additional 4-6 h at room temperature. After completion of reaction and usual work up, the compound Boc protected tetrapeptide (2S ,3S)-benzyl 2-((2S ,5R)-1-((S)-2-((S)-2-(tert-butoxycarbonyl(methyl)amino)propanamido)-2-cyclohexylacetyl)-5-(5-methylfuran-2-yl)pyrrolidine-2-carboxamido)-3 -methylpentano ate (Boc-N(Me)-Ala-Chg-Fro-Ile-OBn) was obtained as yellowish gummy oil in 80% yield (144 mg, 0.2 mmol). HRMS (ESI) (M+H)⁺ calculated for C₄₀H₅₉N₄O₈⁺=723.4327, found 723.4325.

Compound (2S,5S)-benzyl 2-((2S,5R)-1-((S)-2-((S)-2-(tert-butoxycarbonyl(methyl)amino)propanamido)-2-cyclohexylacetyl)-5-(5 -methylfuran-2-yl)pyrrolidine- 2-carboxamido)-3-methylpentano ate (Boc-N(Me)-Ala-Chg-Fro-Ile-OBn) (58 mg, 0.08 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired compound (2S,3S)-benzyl 2-((2S,5R)-1-((S)-2-cyclohexyl-2((S)-2-(methylamino)propanamido)acetyl)-5-(5-methylfuran-2-yl)pyrrolidine-2-carboxamido)-3-methylpentanoate 7 as a white powder in 62% yield (28 mg, 0.05 mmol). The compound 7 was found to be 98% pure at 220 nm on analytical RP-HPLC. ¹H NMR (500 MHz, DMSO-d₆): δ/ppm=8.88 (br, 1H), 8.79 (d, 1H, J=8.3 Hz), 7.86 (d, 1H, J=8.3 Hz), 7.37-7.32 (m, 5H), 6.51 (br d, 1H, J=3.0 Hz), 6.05 (brdd, 1H, J=1.0, 3.0 Hz), 5.34 (m, 1H), 5.17-5.08 (m, 2H), 4.42-4.32 (m, 3H), 3.83-3.79 (m, 1H), 2.50 (s, 3H), 2.24 (s, 3H), 2.09-1.86 (m, 4H), 1.83-1.76 (m, 1H), 1.70-1.41 (m, 7H), 1.33 (d, 3H, J =6.9 Hz), 1.20-1.12 (m, 1H), 1.06-0.92 (m, 3H), 0.86-0.77 (m, 7H), 0.55-0.46 (m, 1H); HRMS (ESI) (M+H)⁺ calculated for C₃₅H₅₁N₄O₆⁺=623.3803, found 623.3800.

### Example 7

Synthesis of (2S,5R)-N-benzhydryl-14(S)-2-cyclohexyl-24(S)-2-(methylamino)propanamido)acetyl)-5-(5-methylfuran-2-yl)pyrrolidine-2-carboxamide as referred in Scheme 3 (Compound 8)

To the solution of intermediate compound IX (62.4 mg, 0.12 mmol) in tetrahydrofuran (1 mL) at −15° C. was added N-methyl morpholine (17.5 μL, 0.16 mmol, 1.5 equiv). After five minutes isobutylchloroformate (17.5 μL, 0.144 mmol, 1. 2 equiv) was added to the reaction mixture. Then after five minutes benzhydrylamine (19 μL, 0.144 mmol, 1.2 equiv) was added and the reaction mixture was stirred for additional 1 h at −15° C. After completion of reaction and usual work up compound Boc protected tetrapeptide tert-butyl (S)-1-((S)-2-((2S ,5R)-2-(benzhydrylcarbamoyl)-5-(5-methylfuran-2-yl)pyrrolidin-1-yl)-1-cyclohexyl-2-oxoethylamino)-1-oxopropan-2-yl(methyl)carbamate was obtained in 67% yield (55 mg, 0.08 mmol). HRMS (ESI) (M +H)⁺ calculated for C₄₀H₅₃N₄O₆⁺=685.3960, found 685.3939.

Compound tert-butyl (S)-1-((S)-2-((2S,5R)-2-(benzhydrylcarbamoyl)-5-(5-methylfuran-2-yl)pyrrolidin-1-yl)-1-cyclohexyl-2-oxoethylamino)-1-oxopropan-2-yl(methyl)carbamate (55 mg, 0.08 mmol) were stirred in 20% TFA/DCM for 30 minutes. After that reaction mixture was concentrated to dryness under reduce pressure. The crude peptide was purified by reversed phase HPLC (RP-HPLC) using C-18 column and then the sample was lyophilized to give the desired compound (2S,5R)-N-benzhydryl-1(S)-2-cyclohexyl-2(S)-2-(methylamino)propanamido)acetyl)-5-(5-methylfuran-2-yl)pyrrolidine-2-carboxamide (8) as a white powder in 62.5% yield (29 mg, 0.05 mmol). The compound 8 was found to be 99% pure at 220 nm on analytical RP-HPLC. ¹H NMR (500 MHz, DMSO-d₆): δ/ppm=8.88 (br, 1H), 8.81 (d, 1H, J=8.0 Hz), 8.30 (d, 1H, J=8.4 Hz), 7.36-7.21 (m, 10H), 6.51 (br d, 1H, J =3.1 Hz), 6.10 (d, 1H, J=8.1 Hz), 6.01 (brdd, 1H, J=1.0, 3.1 Hz), 5.38 (m, 1H), 4.46 (m, 1H), 4.36 (t, 1H, J=8.5 Hz), 3.82 (m, 1H), 2.50 (s, 3H), 2.21-1.89 (m, 4H), 2.14 (s, 3H), 1.70-1.39 (m, 5H), 1.33 (d, 3H, J=7.0 Hz), 1.13-0.89 (m, 4H), 0.79-0.69 (m, 1H), 0.51-0.42 (m, 1H); HRMS (ESI) (M 30 H)⁺ calculated for C₃₅H₄₅N₄⁺=585.3435, found 585.3427.

### Example 8

Synthesis of (2S,5R)-N-2S,3S)-1-(benzhydrylamino)-3-methyl-1-oxopentan-2-yl)-1(S)-2-cyclohexyl-2-((S)-2-(methylamino)propanamido)acetyl)-5-(5- methylfuran-2-yl)pyrrolidine-2-carboxamide (Compound 9)

The (2S,3S)-2-amino-N-benzhydryl-3-methylpentanamide (60 mg, 0.2 mmol) was dissolved in anhydrous DMF (1 mL) and was added to the pre-stirred solution of (2S,5R)-1-((S)-2-((S)-2-(tert-butoxyc arbonyl(methyl)amino)propanamido)-2-cyclohexylacetyl)-5-(5-methylfuran-2-yl)pyrrolidine-2-carboxylic acid (104 mg, 0.2 mmol) and HBTU (75.8 mg, 0.2 mmol) in dry DMF (1 mL) at 0° C. under nitrogen atmosphere followed by addition of DIPEA (0.11 mL, 0.6 mmol). The reaction mixture was further stirred for additional 4-6 h at room temperature. After completion of reaction and usual work up, the compound Boc protected peptide tert-butyl (S)-1-((S)-2-((2S,5R)-2-((2S,3S)-1-(benzhydrylamino)-3 -methyl-1-oxopentan-2-ylcarbamoyl)-5-(5 -methylfuran-2-yl)pyrrolidin-1-yl)-1-cyclohexyl-2-oxoethylamino)-1-oxopropan-2-yl(methyl)carb amate was obtained as brownish gummy oil in 85% yield (135 mg, 0.17 mmol). HRMS (ESI) (M+H)⁺ calculated for C₄₆H₆₄N₅O₇⁺=798.4800, found 798.4817.

Compound tert-butyl (S)-1(S)-2(2S,5R)-2-((2S,3S)-1-(benzhydrylamino)-3-methyl-1-oxopentan-2-ylcarbamoyl)-5-(5-methylfuran-2-yl)pyrrolidin-1-yl)-1-cyclohexyl-2-oxoethylamino)-1-oxopropan-2-yl(methyl)carbamate (80 mg, 0.1 mmol) were stirred in 20% TFA/DCM for 30 minutes. After that, reaction mixture was concentrated to dryness under reduce pressure. The crude peptide was purified by reversed phase HPLC (RP-HPLC) using C-18 column and then the sample was lyophilized to give the desired compound (2S,5R)-N-((2S,3S)-1-(benzhydrylamino)-3-methyl-1-oxopentan-2-yl)-1-((S)-2-c yclohexyl-24(S)-2-(methylamino)prop anamido)acetyl)-5 -(5-methylfuran-2-yl)pyrrolidine-2-carboxamide (9) as a white powder in 60% yield (41 mg, 0.06 mmol). The compound 9 was found to be 98% pure at 220 nm on analytical RP-HPLC. ¹H NMR (500 MHz, DMSO-d₆): δ/ppm=8.92 (d, 1H, J=8.6 Hz), 8.80 (br, 1H), 8.75 (d, 1H, J=8.0 Hz), 7.52 (br d, 1H, J=9.1 Hz), 7.33-7.22 (m, 10H), 6.48 (br d, 1H, J=3.0 Hz), 6.10 (d, 1H, J=8.4 Hz), 6.04 (brdd, 1H, J=1.0, 3.0 Hz), 5.33 (m, 1H), 4.43-4.38 (m, 3H), 3.82 (br m, 1H), 2.50 (s, 3H), 2.26 (s, 3H), 2.14-1.96 (m, 4H), 1.76-1.37 (m, 8H), 1.33 (d, 3H, J =6.8 Hz), 1.09-0.98 (m, 2H), 0.97-0.86 (m, 2H), 0.79-0.72 (m, 7H), 0.54-0.44 (m, 1H); HRMS (ESI) (M+H)⁺ calculated for C₄₁H₅₆N₅O₅⁺=698.4276, found 698.4245.

### Example-9

Synthesis of (2S,5R)-N-42S,3S)-1-(benzhydrylamino)-3-methyl-1-oxopentan-2-yl)-1-(methyl-L-alanyl-L-alanyl)-5-phenylpyrrolidine-2-carboxamide as referred in Scheme 4 (Compound 10)

In a round bottom flash, compound 1-(tert-butyl) 2-methyl (2S,5R)-5-phenylpyrrolidine-1,2-dicarboxylate (3.089 gm, 10.127 mmol) was taken in 50 ml mixture of Methanol/Water (10:1). LiOH (890 mg, 20.255 mmol) was added subsequently in the reaction mixture at 0° C. The reaction mixture was monitored by the TLC. After completion of the reaction, 50 ml of water was added in the reaction, evaporate the organic solvents from the reaction mixture. Subsequently ethyl acetate and water was added, and organic layer was separated out which contain the impurities. Water layer was acidified with citric acid, and ethyl acetate was added in that layer. Organic layer was separated and washed with brine and dried over anhydrous Na₂SO₄. Organic layer was evaporated under reduced pressure to obtain free acid as white solid. Crude acid was directly used for the next step without further purification. NH2-Ile-benzhydrylamide (4.42 gm, 14.948 mmol) was dissolved in dry DCM (15 mL) and was added to the pre-stirred solution of Crude acid (2.9 gm, 9.96 mmol), EDC.HCl (5.71 g, 29.896 mmol) and HOBt (4.035 g, 29.896 mmol) in dry DCM (35 mL) at 0° C. and under nitrogen 15 atmosphere followed by addition of DIPEA (5.17 mL, 29.896 mmol). The reaction mixture was further stirred for additional 4-6 h at room temperature After completion of reaction and usual work up, the crude product was purified by column chromatography using silica gel (40% Ethylacetate/Hexane)) to give title compound (2S,5R)-tert-butyl 2-((2S ,3S)-1-(benzhydrylamino)-3-methyl-1-oxopentan-2-ylcarbamoyl)-5-phenylpyrrolidine-1-carboxylate (XI) as white solid (4.657 gm, 8.184 mmol, yield 82%).

To the compound XI (4.45 gm, 7.82 mmol), 30% TFA/DCM (25 mL) [3m1/mM viz. 1 ml TFA and 2 ml of DCM/mM] were added at 0° C. and reaction was stirred for additional 1 h at room temperature. After that the reaction mixture was concentrated to dryness under reduce pressure and to this DCM (150 mL) was added which was washed with 10% Na₂CO₃ (aq.). The organic layer was dried with anhydrous sodium sulphate and the solvent was removed under reduced pressure, crude amine was afforded as white solid (3.36 gm, 7.036 mmol, yield 90%). This crude amine (600 mg, 1.27 mmol) was dissolved in dry DCM (10 mL) and was added to the pre-stirred solution of Boc-NH-valine (692 mg, 3.195 mmol), EDC.HC1 (1.342 g, 7.034 mmol) and HOBt (950 mg, 7.034 mmol) in dry DCM (25 mL) at 0° C. under nitrogen atmosphere followed by addition of DIPEA (1.22 mL, 7.034 mmol). The reaction mixture was further stirred for additional 4-6 h at room temperature After completion of reaction and usual work up, the crude product was purified by column chromatography using silica gel (40% EA/Hexane) to give title compound tert-butyl (S)-1-((2S ,5R)-2-((2S,3S)-1-(benzhydrylamino)-3-methyl-1-oxopentan-2-ylcarbamoyl)-5-phenylpyrrolidin-1-yl)-3 -methyl-1-oxobutan-2-ylcarbamate (XII) as white solid (723 gm, 1.082 mmol, yield 85%).

To the compound XII (700 mg, 1.047 mmol), 30% TFA/DCM (6 mL) [3 m/mM viz. 1 ml TFA and 2 ml of DCM/mM] were added at 0° C. and reaction was stirred for additional 1 h at room temperature. After that, the reaction mixture was concentrated to dryness under reduce pressure and to this DCM (30 mL) was added which was washed with 10% Na₂CO₃ (aq.). The organic layer was dried with anhydrous sodium sulphate and the solvent was removed under reduced pressure, crude amine was afforded as white solid (740 mg, 0.827 mmol, yield 80%). This crude amine (430 mg, 0.757 mmol) was dissolved in dry DCM (2 mL) and was added to the pre-stirred solution of Boc-val-OH (385 mg, 1.892 mmol), EDC.HCl (1.05 g, 4.163 mmol) and HOBt (563 mg, 4.163 mmol) in dry DCM (8 mL) at 0° C. under nitrogen atmosphere followed by addition of DIPEA (0.97 mL, 4.163 mmol). The reaction mixture was further stirred for additional 4-6 h at room temperature After completion of reaction and usual work up, the crude product was purified by column chromatography using silica gel (40% EA/Hexane) to give title compound tert-butyl (S)-1-((S)-1-((2S,5R)-2-((2S ,3S)-1-(benzhydrylamino)-3-methyl-1-oxopentan-2-ylcarbamoyl)-5-phenylpyrrolidin-1-yl)-3-methyl-l-oxobutan-2-ylamino)-1-oxopropan-2-yl(methyl)carbamate (XIII) as white solid (477 gm, 0.633 mmol, yield 84%).

To the compound XIII (430 mg, 0.571 mmol), 30% TFA/DCM (6 mL) [3 ml/mM viz. 1 ml TFA and 2 ml of DCM/mM] were added at 0° C. and reaction was stirred for additional 1 h at room temperature. After that the reaction mixture was concentrated to dryness under reduce pressure and to this DCM (30 mL) was added which was washed with 10% Na₂CO₃ (aq.). The organic layer was dried with anhydrous sodium sulphate and the solvent was removed under reduced pressure, crude product was purified by column chromatography using silica gel (8% MeOH/DCM) to give title compound (2S,5R)-N-((2S,3S)-1-(benzhydrylamino)-3-methyl-l-oxopentan-2-yl)-1-((S)-3-methyl-2-((S)-2-(methylamino)propanamido)butanoyl)-5-phenylpyrrolidine-2-carboxamide (10) as white solid (326 mg, 0.499 mmol, yield 87%). ESIMS (M+H)⁺ calculated for C₃₉H₅₂N₅O₄=654.4 found 654.6

### Biological Activity

### Example 10: Binding Assays

The SMAC mimetics 1-9 were tested for their ability to displace the fluorescent labeled peptide AVPIAQK(FAM)-OH from XIAP BIR2 or XIAP BIR3 protein. The dose dependent binding experiment were then carried out by sequentially increasing the concentration of SMAC mimetic compounds at constant concentration of fluorescent labeled peptide and XIAP BIR2 or XIAP BIR3 protein. IC₅₋ values were determined from the plot draw by prism software using nonlinear least-squares analysis. The Ki values of the SMAC mimetics were calculated which was based upon the measured IC₅₀ values, the K_{d} value between tracer AVPIAQK(FAM)-OH and XIAP BIR2 or XIAP BIR3 complex, and the concentrations of the protein and tracer in the competition assay by using the web based program which are freely accessed at http://sw16.im.med.umich.edu/software/calc_ki/.In-silico.

SMAC mimetics bind to BIR2 and BIR3 domains of XIAP as determined by FPA. Binding isotherms were plotted between FP reading versus log values of protein concentration in nM. The data was analysed using GraphPad prism and K_{d} values were determined using Boltzmann-sigmoidal non-linear regression curve fitting. First, the Kd values for binding of fluorescent labelled peptide with XIAP BIR2 and XIAP BIR3 were determined to assess the exact Kᵢ values for each molecule bindings towards BIR2/3 domains are shown in Table-1.

TABLE 1
Binding and in-vitro cytotoxic efficacy of Smac mimeties

| | Target Binding | | | | | |
|---|---|---|---|---|---|---|
| | BIR2 | BIR3 | In vitro cytotoxicity at 10 μM dose | | | |
| Compound | Ki (μM) | Ki (μM) | SW 620 | HT29 | HCT116 | VERO |
| 1 | 3.14 | 0.65 | 0.97 ± 2.3 | −0.01 ± 3.6 | −6.59 ± 6.6 | 5.74 ± 3.4 |
| 2 | >10 | >10 | 7.88 ± 8.0 | −1.83 ± 5.5 | −7.06 ± 6.9 | 24.24 ± 22.7 |
| 3 | 1.08 | 0.23 | 1.44 ± 2.1 | −0.46 ± 2.2 | −2.60 ± 6.1 | 3.89 ± 3.4 |
| 4 | 7.39 | 4.38 | −4.10 ± 0.0 | 11.99 ± 0.6 | −8.07 ± 6.6 | 4.54 ± 5.6 |
| 5 | 3.25 | 0.50 | 8.11 ± 11.3 | 40.88 ± 5.3 | 5.96 ± 1.2 | 11.39 ± 1.1 |
| 6 | 3.21 | 0.58 | 59.27 ± 0.1 | 65.03 ± 3.6 | 61.26 ± 2.9 | 18.41 ± 2.1 |
| 7 | >10 | 1.42 | 52.85 ± 22.4 | 71.60 ± 10.2 | 76.51 ± 2.3 | 63.17 ± 5.0 |
| 8 | 1.80 | 0.73 | 38.74 ± 12.6 | 66.04 ± 2.4 | 48.55 ± 6.7 | 39.40 ± 6.9 |
| 9 | >10 | 0.79 | 81.77 ± 3.3 | 72.69 ± 6.9 | 66.15 ± 15.0 | 59.43 ± 11.7 |

### Example 11: Cytotoxicity Assay (SRB Assay)

In vitro cytotoxic activities of different compounds were assessed by using standard SRB assay in different cells. The absorbance of the treated and untreated cells was measured on a multi-well scanning spectrophotometer (Epoch Microplate Reader, Biotek, USA) at a wavelength of 510 nm. Percent growth inhibition was calculated by using the formula [100-(Absorbance of compound treated cells/Absorbance of untreated cells)] X 100.

SMAC mimetics promotes tumor cell selective cytotoxic effects. SW 620, HT 29, HCT 116 and VERO cells were treated with series of Smac mimetics at 10 μM dose for 48 hours and cytotoxicity was measured by SRB assay. Percent growth inhibition was tabulated in Table 1 as provided above. As C6 had shown robust in-vitro cytotoxic effect against tumor cells but not to VERO cells, we determined IC₅₀ value of C6 against different types of cancer cell lines. ICso values are represented in Table 2. We also assessed In Silico Molecular Docking Analysis of C6 (FIG. 1) and observed that contributing residues responsible for the stabilization of C-6 on BIR2 shows the active role of S162, E163 and R166 via hydrogen bonds while Y161 and F229 making base stacking interactions. Furthermore, the analysis of binding pocket of BIR3 displays that Y324, R299 and G306 are forming the hydrogen bonds and W323 residues shows base stacking interaction in the stabilization of C6 compound.

TABLE-2
Cytotoxic activity of C6 against various cells

| Cancer Type | Cell Line | IC₅₀ (μM) |
|---|---|---|
| Colon | HT29 | 6.66 |
| Colon | SW620 | 4.31 |
| Colon | HCT116 | 8.93 |
| Colon (Mouse) | CT26 | >10 |
| Breast | MDAMB231 | 4.15 |
| Breast | HCC1806 | 5.79 |
| Breast | MCF-7 | 5.02 |
| Breast (Mouse) | 4T1 | 4.17 |
| Lung | A-549 | >10 |
| Kidney | 786-O | 7.2 |
| Prostrate | PC-3 | 6.02 |
| Brain | A172 | 6.88 |
| Ovary | SK-OV-3 | 3.91 |
| Pancreas | PANC-1 | 5.83 |
| Liver | HEP-G2 | 7.3 |
| Melanoma (Mouse) | B-16 | 6.7 |
| Leukemia | K562 | 9.10 |
| Lymphoma | U937 | 8.98 |
| Kidney (monkey) | VERO | >10 |

### Example 12: Apoptosis Antibody Array Analysis

Apoptosis array was performed by using Proteome Profiler Human Apoptosis Array Kit (ARY009) from R&D Systems following the manufacturer's instructions. The detailed assay procedure was followed. The images were captured by the gel documentation system (Bio-Rad chemidoc XRS plus), while ImageJ software (NIH) was used for analysis and quantification. Plotly software was used for heatmap generation (Montreal, Canada).

It was observed that compound C6 promotes smac driven apoptotic features as observed by right shift of histogram overlays show the Annexin-V positive cells (FIG. 2, left).

Further, C6 treatment drives SMAC mediated hall mark apoptotic features as observed by cleavage of PARP and Caspase-3 and degradation of cIAP-1 in treated cells as compared to control (FIG. 2, right and FIG. 3). Further, C6 robustly sensitizes TRAIL mediated tumor cell cytotoxic response in vitro (FIG. 4). Inhibition of apoptosis or Caspases or overexpression of its target like XIAP or knocking down of DR5 rescues C6 mediated cell death.

### Example 13: Colony Formation Assay

The clonogenic colony formation assay was done on single-cell suspension. Briefly, cells were plated in complete McCoy's medium into 12-well plates and 24 hours later, were treated with different agents at different doses either alone or in combination. The cells were cultured for two weeks with renewing the media every 3^{rd} day. The plates were washed with PBS and fixed with ice-cold methanol followed by staining with 0.5% crystal violet in methanol for 30 min. Excess stain was removed by washing with water thoroughly and plates were allowed to dry. Representative images were captured in the gel documentation system (Bio-Rad chemidoc XRS plus) while ImageJ software (NIH) was used for analysis and quantification to monitor single cell colony formation efficiency under the different treatment combinations.

Inhibition of apoptosis by Pan caspase inhibitor Z-VAD-FMK markedly rescues cytotoxic phenotype of C6 as observed by colony formation assay and confirmed that C6 induces apoptotic cell death in cancer cells (FIG. 5, middle top panel). Similarly, colony formation assay in XIAP overexpressed and DR5 knock down cells showed significant resistant of C6 mediated apoptotic cell death suggesting the critical involvement of XIAP and DR5 in the whole apoptotic process (FIG. 5 lower left and right panels)

### Example 14: In Vivo Studies in Xenograft Tumor Models

All the animals were maintained in a pathogen-free facility under a day—night cycle.

Following our well-established colon cancer xenograft models, 2×10⁶ cells (SW 620 and HCT 116) or 0.5×10⁶ cells (HCT 116) in 100 μl PBS were subcutaneously inoculated into the flanks of the left/and or right hind leg of each 4-6 weeks old nude Crl: CD1-Foxnlnu mice. Mice were randomly assigned to groups by a blinded independent investigator. Throughout the study, the tumor was measured with an electronic digital caliper at a regular interval, and the tumor volume was calculated using standard formula V=Π/6×a2×b, where ‘a’ is the short and ‘b’ is the long tumor axis. At the end of the experiment, mice were sacrificed, and subcutaneous tumors were dissected for further studies. Parts of harvested tumors were minced into small pieces with sterile forceps and scissors and homogenized for lysate preparation.

Utilizing the cisplatin resistant SW-620 xenograft model, we determined the in vivo efficacy of C6 and observed that it has potent anti tumor efficacy against the same model (FIG. 7). Similarly, it was observed that C6 subcutaneous and oral administration markedly reduced the HCT-116 xenograft tumor volume and weight as compared to respective controls. (FIG. 8). Immunoblots from tumor tissues reveal that C6 treated tumors show reduced XIAP and cIAP1 protein expression as compared to vehicle treated tumors in vivo, which confirms that C6 targets these proteins and reduces the tumor volume (FIG. 9, left panel). Further, in our organ distribution analysis, we observed presence of C6 in tumor tissues in significant quantity confirming the delivery of the molecule to the tumor site (FIG. 9, right panel).

### Example 15: Stability and Pharmacokinetics Studies of C6

SIF, SGF and other stability studies were performed by following standard protocol. LC-MS/MS method was developed for C6: Intravenous group (C6, 4 mg/kg), Subcutaneous group (C6, 30 mg/kg), Oral group (C6, 30 mg/kg). Mice were administered their respective doses according to body weight by intravenous (lateral vein), Subcutaneous route and Oral respectively. Blood samples were collected at 0.083, 0.25, 0.5, 0.75, 1, 2, 4, 8, 12, 24 and 48 hours. Plasma was separated and processed for analysis. For pharmacokinetic analysis, plasma concentration versus time data were plotted and analyzed by non-compartmental analysis method using WinNonlin (Pharsight, Mountain View, CA) software.

C6 is quite stable in SIF, SGF, plasma, MLM, HLM as shown in FIG. 6. The pharmacokinetic profile of C6 is by IV, SC and Oral route are given in figure and pharmacokinetic parameters are listed in Table 3. The absolute bioavailability of C6 by subcutaneous route and oral route was found 56.61±7.21% and 55.93±11.15% respectively as presented in Table-3.

TABLE 3
Pharmacokinetic properties of C6

| Parameter (Unit) | IV route | SC route | Oral route |
|---|---|---|---|
| Cₘₐₓ (ng/mL) | 10215 ± 2633.37 | 5018.33 ± 853.66 | 4490.00 ± 1514.05 |
| AUC_{0-∞} (h * ng/mL) | 4107.24 ± 852.34 | 17439.13 ± 2220.28 | 17227.95 ± 3433.39 |
| Tₘₐₓ (h) | 0.08 | 0.88 ± 0.21 | 1.08 ± 0.47 |
| T_{1/2} (h) | 0.19 ± 0.05 | 2.10 ± 0.15 | 3.84 ± 1.80 |
| C1 (L/hr/Kg) | 1.02 ± 0.24 | 1.75 ± 0.24 | 1.79 ± 0.32 |
| Vd (L/Kg) | 0.28 ± 0.12 | 5.32 ± 0.98 | 9.52 ± 3.75 |
| MRT (h) | 1.72 ± 0.46 | 8.92 ± 1.35 | 4.18 ± 1.81 |
| Absolute | — | 56.61 ± 7.21 | 55.93 ± 11.15 |
| Bioavailability (%) | | | |

1-14. (canceled)

15. A SMAC mimetic compound of Formula-I, 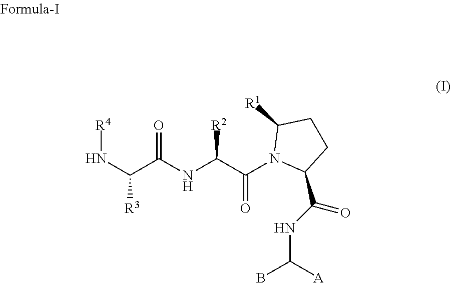
wherein,
R¹ is selected from the group consisting of hydrogen, and unsubstituted or substituted heteroaryl or C₆-C₁₀ aryl;
R² is selected from the group consisting of H, Ci₁-C₆ alkyl and C₄-C₈ cycloalkyl;
R³ and R⁴ are each independently selected from the group consisting of H and C₆-C₁₀ alkyl;
A is selected from unsubstituted or substituted C₆-C₁₀ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof.

16. The SMAC mimetic compound as claimed in claim 15, selected from the group consisting of
L-alanyl-L-valyl-L-prolyl-L-isoleucine (compound 1),
benzyl ((2S,5R)-1-(L-alanyl-L-valyl)-5-(5-methylfuran-2-yl)pyrrolidine-2-carbonyl)-L-isoleucinate (compound 2),
(2S,5R)-1-(L-alanyl-L-valyl)-N-benzhydryl-5-(5-methylfuran-2-yl)pyrrolidine-2-carboxamide (compound 3),
(2S,3S)-benzyl 3-methyl-2-((2S,5R)-1-((S)-3-methyl-24(S)-2-(methylamino)propanamido)butanoyl)-5-(5-methylfuran-2-yepyrrolidine-2-carboxamido)pentanoate (compound 4),
(2S,5R)-N-benzhydryl-1-(methyl-L-alanyl-L-valyl)-5-(5-methylfuran-2-yl)pyrrolidine-2-carboxamide (compound 5),
(2S,5R)-N-((2S,3S)-1-(benzhydrylamino)-3-methyl-1-oxopentan-2-yl)-1-(methyl-L-alanyl-L-valyl)-5-(5-methylfuran-2-yl)pyrrolidine-2-carboxamide (compound 6),
(2S,3S)-benzyl 2-((2S,5R)-1-((S)-2-cyclohexyl-2-((S)-2-(methylamino)propanamido)acetyl)-5-(5 -methylfuran-2-yl)pyrrolidine-2-carboxamido)-3-methylpentanoate (compound 7),
(2S,5R)-N-benzhydryl-1(S)-2-cyclohexyl-2-((S)-2-(methylamino)propanamido)acetyl)-5-(5-methylfuran-2-yepyrrolidine-2-carboxamide (Compound 8),
(2S,5R)-N-((2S,3S)-1-(benzhydrylamino)-3-methyl-1-oxopentan-2-yl)-14(S)-2-cyclohexyl-2-((S)-2-(methylamino)propanamido)acetyl)-5-(5-methylfuran-2-yl)pyrrolidine-2-carboxamide (Compound 9); and
(2S,5R)-N-((2S,3S)-1-(benzhydrylamino)-3-methyl-1-oxopentan-2-yl)-1-(methyl-L-alanyl-L-alanyl)-5-phenylpyrrolidine-2-carboxamide (Compound 10).

17. A process for preparation of SMAC mimetics compound of Formula-I, 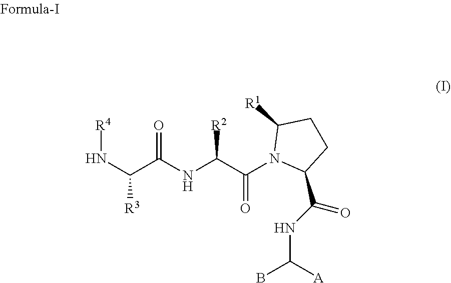
wherein,
R¹ is selected from the group consisting of hydrogen, and unsubstituted or substituted heteroaryl or C₆-C₁₀ aryl;
R² is selected from the group consisting of H, C₁-C₆ alkyl and C₄-C₈ cycloalkyl;
R³ and R⁴ are each independently selected from the group consisting of H and C₁-C₈ alkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, C-C₆ alkoxy and C₆ alkoxyaryl;
R⁶ and R⁷ are each independently selected from the group consisting of hydrogen, C₆-C₁₀ aryl and C₆-C₁₀ arylalkyl;
comprising the steps of,
i. removing the Boc-group of 2-benzyl 1-(tert-butyl) (2S,5S)-5-(5-methylfuran-2-yl)pyrrolidine-1,2-dicarboxylate by acidolysis using TFA followed by coupling of resulting amine with BocNHCH(R^{2′})COOH in presence of a peptide coupling reagent and a weak base to obtain a compound of formula P1; 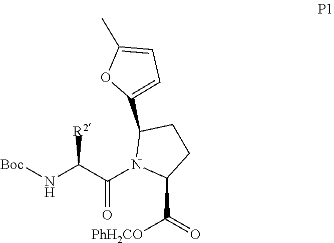
ii. removing Boc group from the compound of formula P1 by acidolysis using TFA and coupling the resulting amine with a compound of formula BocN(R^{4′})CH(R^{3′})COOH in presence of a peptide coupling reagent and a weak base to obtain a compound of formula P2; 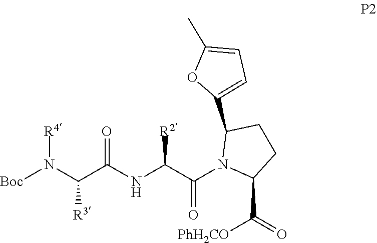
iii. catalytic hydrogenation of the compound of formula P2 using Pd-catalyst in presence of a solvent to obtain a free carboxylic acid of formula P3; 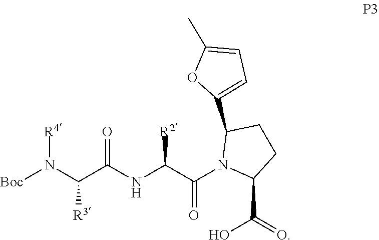
iv. coupling the free carboxylic acid of formula P3 with NH2CH(A)(B) in presence of a peptide coupling reagent and a weak base to obtain the compound of formula I.

18. The process as claimed in claim 17, wherein the peptide coupling reagent is selected from the group consisting of HOBt, EDCI and HBTU, wherein the weak base is diethylisopropylamine and the Pd-catalyst is selected from Pd/C, or Pd(OH)₂/C.

19. The process as claimed in claim 17, wherein the solvent is selected from DCM, or DMF for peptide coupling and MeOH, or EtOAc for catalytic hydrogenation.

20. A process of preparation of SMAC mimetic compound 10 as claimed in claim 16, comprising the steps of:
(a) saponification and coupling of a compound X with H-Ile-benzhydryl amide in presence of a peptide coupling reagent and a weak base in a solvent to obtain a compound XI; 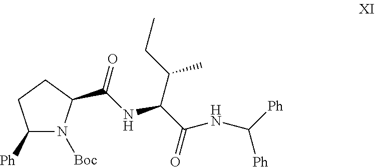 [SMILES: CC[C@H](C)[C@H](NC(=O)[C@@H]1CC[C@H](c2ccccc2)N1C(=O)OC(C)(C)C)C(=O)NC(c1ccccc1)c1ccccc1]
(b) removing the Boc-group from the compound XI by acidolysis using TFA followed by coupling of the resulting amine with Boc-Val-COOH in the presence of a peptide coupling reagent and a weak base to obtain a compound of formula XII; 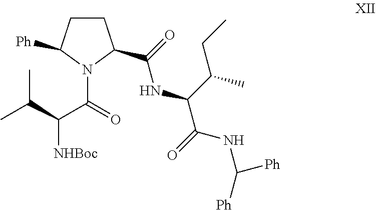 [SMILES: CC[C@H](C)[C@H](NC(=O)[C@@H]1CC[C@H](c2ccccc2)N1C(=O)[C@@H](NC(=O)OC(C)(C)C)C(C)C)C(=O)NC(c1ccccc1)c1ccccc1]
(c) removing the Boc-group from the compound XII by acidolysis using TFA followed by coupling of the resulting amine with Boc-N-Me-Ala-OH in the presence of a peptide coupling reagent and a weak base to obtain a compound of formula XIII; and 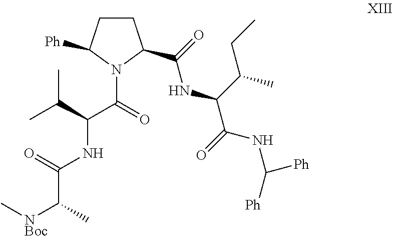 [SMILES: CC[C@H](C)[C@H](NC(=O)[C@@H]1CC[C@H](c2ccccc2)N1C(=O)[C@@H](NC(=O)[C@H](C)N(C)C(=O)OC(C)(C)C)C(C)C)C(=O)NC(c1ccccc1)c1ccccc1]
(d) removing the Boc-group from the compound of formula XIII by acidolysis to obtain the compound 10

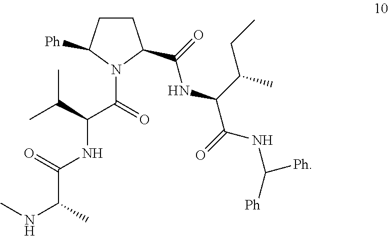 [SMILES: CC[C@H](C)[C@H](NC(=O)[C@@H]1CC[C@H](c2ccccc2)N1C(=O)[C@@H](NC(=O)[C@H](C)NC)C(C)C)C(=O)NC(c1ccccc1)c1ccccc1]

21. The process as claimed in claim 20, wherein the peptide coupling reagent is selected from the group consisting of HOBt, EDCI and HBTU, wherein the solvent is selected from DCM, or DMF and the weak base is diethylisopropylamine and for acidolysis the reagent is TFA.

22. The SMAC mimetic compound as claimed in claim 15, wherein the compound inhibits binding of Smac protein to Inhibitor of Apoptosis Proteins (IAPB) and is useful in treatment of proliferative diseases including cancer.

23. A pharmaceutical composition comprising SMAC mimetic compound of Formula-I, 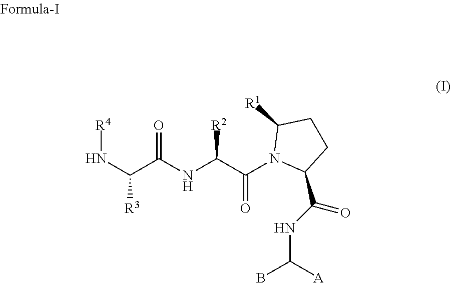
wherein,
R¹ is selected from the group consisting of hydrogen, and unsubstituted or substituted heteroaryl or C₆-C₁₀ aryl;
R² selected from the group consisting of H, C₆-C₁₀ alkyl and C₄-C₈ cycloalkyl and C₄-C₈ cycloalkyl;
R³ and R⁴ are each independently selected from the group consisting of H and C₁-C₆ alkyl;
A is selected from unsubstituted or substituted C₁-C₆ alkyl or C₆-C₁₀ aryl;
B is selected from the group consisting of C₆-C₁₀ aryl, C(O)R⁵ and C(O)N(R⁶)(R⁷);
R⁵ is selected from the group consisting of OH, C₁-C₆ alkoxy and C₆ alkoxyaryl;
R⁶ and R⁷ are each independently selected from the group consisting of hydrogen, C₆-C₁₀ aryl and C₆-C₁₀ arylalkyl;
or a pharmaceutically acceptable salt thereof and a pharmaceutically acceptable excipient.

24. The SMAC mimetic compound as claimed in claim 15 having potent anti-proliferative activity against mammalian cancer cell lines selected from the group consisting of colon, breast, kidney, prostate, brain, ovary, pancreas, melanoma, liver, leukemia and lymphoma.

25. The SMAC mimetic compound as claimed in claim 15, wherein the compound is useful in treatment of therapy resistant, refractory, and metastatic cancers in mammals.

26. The SMAC mimetic compound as claimed in claim 15, wherein the compound is useful in combination therapies with other anti-proliferative agents selected from the group consisting of TRAIL agonists/MAbs, aromatase inhibitors, epigenetic modulators, kinase inhibitors, alkylating agents, microtubule disrupters, topoisomerase inhibitors, antiangiogenic compounds, Hsp90 inhibitors, mTOR inhibitors, estrogen and androgen antagonists, MMP inhibitors and biological response modifiers.

27. A method for treating cancer using SMAC mimetic compounds as claimed in claim 15.
